(19) United States
(12) Patent Application Publication
(10) Pub. No.: US 2017/0247446 A1
(43) Pub. Date: Aug. 31, 2017

(54) TARGETING K-RAS-MEDIATED SIGNALING PATHWAYS AND MALIGNANCY BY ANTI-hLIF ANTIBODIES

(71) Applicant: The Regents of the University of California, Oakland, CA (US)

(72) Inventors: Frank MCCORMICK, San Francisco, CA (US); Man-Tzu WANG, San Francisco, CA (US)

(21) Appl. No.: 15/508,036

(22) PCT Filed: Sep. 10, 2015
(86) PCT No.: PCT/US2015/049461

§ 371 Date: Mar. 1, 2017

Related U.S. Application Data
(60) Provisional application No. 62/048,770, filed on Sep. 10, 2014.

(51) Int. Cl. C07K 16/24; A61K 39/395; A61K 31/7068

(52) U.S. Cl. CPC C07K 16/244; A61K 31/7068; A61K 39/39558; C07K 2317/76

(57) ABSTRACT

The present invention provides method of treating a K-Ras-expressing cancer in a subject comprising administering to the subject a therapeutic amount of an agent that antagonizes leukemia inhibitory factor (LIF). Compositions and kits for treating a K-Ras-expressing cancer in a subject are also provided.

### CROSS-REFERENCES TO RELATED APPLICATIONS

This application claims priority to U.S. Provisional Application No. 62/048,770, filed Sep. 10, 2014, the entire content of which is incorporated by reference herein for all purposes.

### BACKGROUND OF THE INVENTION

Pancreatic cancer is a cancer that often has a poor prognosis, even when detected in its early stages. It is estimated that for all stages of pancreatic cancer combined, only 6% of patients survive five years after diagnosis. The most common form of pancreatic cancer, pancreatic ductal adenocarcinoma (PDAC), is known to have an extremely poor prognosis. Although survival time improves for patients who undergo a surgical resection, PDAC frequently is not diagnosed in time for surgical resection to be feasible.

The oncogene K-Ras is frequently mutated in cancers, such as pancreatic, lung, and colorectal cancers, with activating K-Ras mutations present in over 90% of PDACs. However, to date there have been no successes in developing small molecule inhibitors that directly block K-Ras function and show efficacy in pre-clinical model.

### BRIEF SUMMARY OF THE INVENTION

In one aspect, methods of treating a cancer in a subject are provided. In some embodiments, the method comprises administering to the subject a therapeutic amount of an agent that antagonizes leukemia inhibitory factor (LIF).

In some embodiments, the cancer is a K-Ras-expressing cancer. In some embodiments, the K-Ras-expressing cancer is a cancer that expresses wild-type K-Ras. In some embodiments, the K-Ras-expressing cancer is a cancer that expresses a mutated K-Ras.

In some embodiments, the cancer is a pancreatic cancer, a colorectal cancer, or a lung cancer. In some embodiments, the cancer is pancreatic cancer (e.g., pancreatic ductal adenocarcinoma).

In some embodiments, the agent that antagonizes LIF is an anti-LIF antibody. In some embodiments, the anti-LIF antibody is a monoclonal antibody. In some embodiments, the anti-LIF antibody is an antibody fragment selected from the group consisting of a Fab, a F(ab′)₂, and a Fv.

In some embodiments, the agent that antagonizes LIF is administered orally, intravenously, or intraperitoneally.

In some embodiments, the agent that antagonizes LIF is administered in combination with a chemotherapeutic agent. In some embodiments, the chemotherapeutic agent is a nucleoside analog. In some embodiments, the chemotherapeutic agent is gemcitabine. In some embodiments, the agent that antagonizes LIF and the chemotherapeutic agent are administered concurrently. In some embodiments, the agent that antagonizes LIF is administered and the chemotherapeutic agent are administered sequentially.

In another aspect, compositions and kits for treating a cancer are provided. In some embodiments, the composition or kit comprises:

an agent that antagonizes leukemia inhibitory factor (LIF); and

a chemotherapeutic agent.

In some embodiments, the cancer is a K-Ras-expressing cancer. In some embodiments, the K-Ras-expressing cancer is a cancer that expresses wild-type K-Ras. In some embodiments, the K-Ras-expressing cancer is a cancer that expresses a mutated K-Ras. In some embodiments, the cancer is a pancreatic cancer, a colorectal cancer, or a lung cancer. In some embodiments, the cancer is pancreatic cancer (e.g., pancreatic ductal adenocarcinoma).

In some embodiments, the chemotherapeutic agent is a nucleoside analog. In some embodiments, the chemotherapeutic agent is gemcitabine.

In another aspect, compositions comprising an agent that antagonizes LIF for use in treating a cancer are provided. In some embodiments, the cancer is a K-Ras-expressing cancer (e.g., a cancer that expresses wild-type K-Ras or a cancer that expresses a mutated K-Ras). In some embodiments, the cancer is a pancreatic cancer, a colorectal cancer, or a lung cancer. In some embodiments, the composition comprising an agent that antagonizes LIF is used in combination with a chemotherapeutic agent. In some embodiments, the composition comprising an agent that antagonizes LIF further comprises a chemotherapeutic agent. In some embodiments, the chemotherapeutic agent is gemcitabine.

In still another aspect, the use of a composition comprising an agent that antagonizes LIF for the manufacture of a medicament for the treatment of a cancer is provided. In some embodiments, the cancer is a K-Ras-expressing cancer (e.g., a cancer that expresses wild-type K-Ras or a cancer that expresses a mutated K-Ras). In some embodiments, the cancer is a pancreatic cancer, a colorectal cancer, or a lung cancer. In some embodiments, the composition comprising an agent that antagonizes LIF further comprises a chemotherapeutic agent. In some embodiments, the chemotherapeutic agent is gemcitabine.

### BRIEF DESCRIPTION OF THE DRAWINGS

FIG. 1. LIF-pSTAT3 signaling is regulated by oncogenic K-Ras in pancreatic cancer. (A) Microarray analysis (affymetrix Gene ST1.0) revealed that LIF was significantly unregulated in K-Ras^{V12}-transformed NIH/3T3 cells when compared to vector control or H-Ras^{V12}-transformed one. (N=3). (B) qPCR (left panel) and Western blotting analysis (right panel) confirmed the elevated LIF expressions for mRNA and protein in K-Ras^{V12}-transformed NIH/3T3 cells. (Right panel) The increased LIF expression referred to enhanced phosphorylated levels of STAT3 in K-Ras^{V12}-transformed. (N=3 in qPCR analysis; ** P<0.01; *** P<0.001). (C) qPCR analysis suggested that mutant K-Ras driven mouse pancreatic cancers had higher LIF expression when compared to mutant B-Raf induced mouse pancreatic tumors. (N=3; ** P<0.01). (D) LIF/LIFR expressions in pancreatic cancer tissues and other types of cancers according to K-Ras mutation status. (E) LIF expressions in established pancreatic cancer cell lines are uncorrelated to specific K-Ras mutation isoforms.

(F) Knock-down LIF by shRNA suppressed sphere forming ability in oncogenic K-Ras driven mPCACs (N=8, *** P<0.0001). (G) In two different lines of oncogenic K-Ras driven mPCACs, knock-down of LIF dramatically reduced the recurrent colony forming efficiency post-5FU treatments when compared to control cells. (H) Knock-down of LIF reduced the formation of macro-metastatic spleen lesions and increased media survival time in oncogenic K-Ras driven mPDACs.

FIG. 2. LIF-pSTAT3 signaling is regulated by oncogenic K-Ras in pancreatic cancer. (A-E) LIF expression levels are regulated by K-Ras in pancreatic cancer cell lines. (A) PANC2.13 with knock-down expression of K-Ras showed reduced expressions of LIF and phospho-STAT3 from Western blot. (B) PANC1.0 and (C) PANC2.03 with knock-down expression of K-Ras showed reduced expressions of LIF and phospho-STAT3 at mRNA levels. (N=3; * P<0.05; *** P<0.001). (D) CaPanI and (E) HcG25 with knock-down expression of K-Ras showed reduced expressions of LIF and phospho-STAT3 at mRNA levels. (N=3; ** P<0.01; *** P<0.001; **** P<0.0001). (F) LIF ELISA revealed that human pancreatic cancer cell lines with knock-down expression of K-Ras secreted significantly decreased LIF in culture media in the comparison to control cells. (N=4; * P<0.05; **** P<0.00001). (G) Western blot suggested that the pancreatic cancer cells, with knock-down expression of K-Ras and sequentially down-regulated expression of LIF, showed decreased phospho-STAT3 levels. (H) phospho-STAT luciferase reporter assays suggested that the pancreatic cancer cells with knock-down expression of K-Ras had significantly decreased STAT3 transcriptional activity. (N=3; ** P<0.01; *** P<0.001).

FIG. 3. LIF plays important roles in human pancreatic cancer growth/initiation. (A) Western blot to confirm the knock-down efficiency of shRNA targeting human LIF in human pancreatic cancer cell lines. (B) Tumor free survival curve of PANC2.03 in subcutaneous xenograft model suggested that the cancer cells with knock-down expression of LIF possessed dramatically reduced tumor-initiating ability, when compared to control cells. (N=6). (C) The pancreatic tumors in subcutaneous xenograft with knock-down expression of LIF grew in a significantly slow rate when compared to control tumors. (N=6; * P<0.01). (D) Knock-down of LIF expression reduced tumor initiation rate in the PANC1-driven tumors in orthotopic model. (N=4).

FIG. 4. LIF is required for resistance to gemcitabine treatments in pancreatic cancers. An MTS ((3-(4,5-dimethylthiazol-2-yl)-5-(3-carboxymethoxyphenyl)-2-(4-sulfophenyl)-2H-tetrazolium) assay, which is a colorimetric assay for assessing cell viability, suggested that knock-down of LIF sensitized PANC2.03 cells to gemicitabine treatment when compared to control cells. (N=6) Vehicle treatment (DMSO) was used for normalization.

FIG. 5. LIF neutralizing antibody prevents tumor initiation and improves therapeutic efficacy of gemcitabine in pancreatic cancer cells. (A) LIF ELISA with anti-hLIF antibody competition assay confirmed the neutralizing abilities of our target antibodies. (B) 50,000 cells of PANC2.03 were subcutaneously injected into nude mice for the tumor initiation assay. The anti-LIF AB (clone D25.1.4) was given before the tumor inoculation at 10 mg/kg. The treatment was given three times a week. As shown, LIF antibodies dramatically prevent tumor initiation. (C) In drug sensitization assay, 0.2×10⁶ cells of PANC2.03 cells were first subcutaneously injected into nude mice. The tumors were formed within 14 days post-inoculation. The mice were randomly separated into 4 different groups: control, Ab only, gemcitabine only, and combination. The combination treatment of gemcitabine and LIF Ab caused the complete regressions in 8 out of 10 tumors, whereas control IgG, LIF antibody (D25.1.4) alone, or gemcitabine alone did not lead to the tumor regressions. (D) Tumor volume changing curve in the PANC2.03 subcutaneous tumors with the treatment model of (C). (E) The combination treatment of gemcitabine and LIF Ab dramatically reduced the tumor proliferation rate, whereas the tumors treated with gemcitabine alone still had a positive growth rate. Tumor proliferation rate=(Tumor vol on the later day−Tumor vol on the initial day)/Tumor vol on the initial day*100. (F) Fold change on tumor volumes suggested that the combination treatment of gemcitabine and LIF Ab dramatically reduced the tumor proliferation rate, whereas the tumors treated with gemcitabine alone still had a positive growth rate.

FIG. 6. LIF expression is enriched in multiple types of cancers. (A-I) The online software Oncomine™ (Invitrogen) was used to analyze different published datasets to determine LIF expression levels for multiple types of cancers as compared to normal tissues. (A-B) LIF expression in TCGA colorectal cancer dataset. (C) LIF expression in D'Errico gastric cancer dataset. (D) LIF expression in Wang gastric cancer dataset. (E) LIF expression in Bredel brain cancer dataset. (F) LIF expression in Barretina cell line dataset. LIF expression was found to be enriched in pancreatic cancer. (G) LIF expression in Garnett cell line dataset. LIF expression was found to be enriched in pancreatic cancer. (H) LIF expression in Pei pancreatic cancer dataset. (I) LIF expression in Garnett cell line dataset. LIF expression at mRNA was significantly enhanced in the established cancer cell lines with mutant K-Ras when compared to cancer cell lines with wild-type K-Ras expression.

FIG. 7. LIF expression at mRNA is dramatically decreased in chemotherapy-sensitive cancers. (A-I) The online software Oncomine™ (Invitrogen) was used to analyze different published datasets with gene profiles of chemotherapy-sensitive and -resistant tumor specimens to determine LIF expression levels. (A) LIF expression in Garnett cell line dataset (cytarabine-resistant and cytarabine-sensitive brain and CNS cancer cell lines). (B) LIF expression in Garnett cell line dataset (vorinostat-resistant and vorinostat-sensitive multi-cancer cell lines). (C) LIF expression in Garnett cell line dataset (AZD8055-resistant and AZD8055-sensitive brain and CNS cancer cell lines). (D) LIF expression in Garnett cell line dataset (tretinoin-resistant and tretinoin-sensitive brain and CNS cancer cell lines).

### DETAILED DESCRIPTION OF THE INVENTION

### I. Introduction

The present invention is based in part on the surprising discovery that leukemia inhibitor factor (LIF), a stem cell and STAT3 regulated chemokine that is highly expressed in human pancreatic cancer cell lines, is regulated by oncogenic K-Ras. Without being bound to a particular theory, it is believed that LIF acts as a downstream effector essential for K-Ras-driven pancreatic cancer by regulating the stemness of pancreatic cancer cells with activated K-Ras.

Accordingly, in one aspect the invention provides methods of treating a cancer, such as a cancer that expresses wild-type K-Ras or a cancer that expresses a mutated K-Ras, in a subject by administering a therapeutic amount of an agent that antagonizes LIF. In another aspect, the invention also provides compositions and kits for treating a cancer, such as a K-Ras-expressing cancer, comprising an agent that antagonizes LIF, optionally in combination with a chemotherapeutic agent.

### II. Definitions

As used herein, the term “K-Ras” refers to “Kirsten rat sarcoma viral oncogene homolog.” The protein encoded by the K-Ras gene is a small GTPase that functions in intracellular signal transduction. Human K-Ras gene and protein sequences are set forth in, e.g., Genbank Accession Nos. M54968.1 and AAB414942.1. Some common K-Ras genes and proteins found in human cancers contain mutations at codon 12, codon, codon 61, codon 146, and/or other concurrent sites. Non-limiting examples of K-Ras mutations include mutations at codon 5 (e.g., K5E), codon 9 (e.g., V9I), codon 12 (e.g., G12A, G12C, G12D, G12F, G12R, G12S, G12V, G12Y), codon 13 (e.g., G13C, G13D, G13V), codon 14 (e.g., V141, V14L), codon 18 (e.g., A18D), codon 19 (e.g., L19F), codon 22 (e.g., Q22K), codon 23 (e.g., L23R), codon 24 (e.g., I24N), codon 26 (e.g., N26K), codon 33 (e.g., D33E), codon 36 (e.g., I36L, I36M), codon 57 (e.g., D57N), codon 59 (e.g., A59E, A59G, A59T), codon 61 (e.g., Q61H, Q61K, Q61L, Q61R), codon 62 (e.g., E62G, E62K), codon 63 (e.g., E63K), codon 64 (e.g., Y64D, Y64H, Y64N), codon 68 (e.g., R68S), codon 74 (e.g., T74P), codon 92 (e.g., D92Y), codon 97 (e.g., R97I), codon 110 (e.g., P110H, P110S), codon 117 (e.g., K117N), codon 118 (e.g., C118S), codon 119 (e.g., D119N), codon 135 (e.g., R135T), codon 138 (e.g., G138V), codon 140 (e.g., P140H), codon 146 (e.g., A146T, A146V), codon 147 (e.g., K147N), codon 153 (e.g., D153N), codon 156 (e.g., F156L), codon 160 (e.g., V160A), codon 164 (e.g., R164Q), codon 171 (e.g., I171M), codon 176 (e.g., K176Q), codon 185 (e.g., C185R, C185S), and codon 188 (e.g., M188V).

A “K-Ras-expressing cancer” refers to a cancer that has a detectable level of expression of K-Ras (either wild-type or its mutant forms). In some embodiments, a cancer has a detectable level of expression when at least 0.1% of cells in the cancer tissue sample are positive for K-Ras activation (e.g., wild-type K-Ras or a K-Ras activating mutation at codon 12, codon 13, codon 61, and/or other codons). In some embodiments, the cancer has a detectable level of expression of wild-type K-Ras. In some embodiments, the cancer has a detectable level of expression of a mutated K-Ras. In some embodiments, a K-Ras-expressing cancer has a level of expression of K-Ras (e.g., wild-type K-Ras or mutated K-Ras) that is at least 5%, 10%, 20%, 30%, 40%, 50%, 75%, 100%, 150%, or 200% greater than the level of K-Ras expression in a control (e.g., a non-diseased cell or tissue that does not express K-Ras, such as normal human peripheric lymphocytes).

The term “cancer” refers to a disease characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of different types of cancer include, but are not limited to, digestive and gastrointestinal cancers such as gastric cancer (e.g., stomach cancer), colorectal cancer, gastrointestinal stromal tumors, gastrointestinal carcinoid tumors, colon cancer, rectal cancer, anal cancer, bile duct cancer, small intestine cancer, and esophageal cancer; breast cancer; lung cancer; gallbladder cancer; liver cancer; pancreatic cancer; appendix cancer; prostate cancer, ovarian cancer; renal cancer; cancer of the central nervous system; skin cancer (e.g., melanoma); lymphomas; gliomas; choriocarcinomas; head and neck cancers; osteogenic sarcomas; and blood cancers. As used herein, a “tumor” comprises one or more cancerous cells. In some embodiments, the cancer is pancreatic cancer.

“Leukemia inhibitory factor (LIF)” refers to an interleukin class 6 cytokine that inhibits cell differentiation. Human LIF gene and protein sequences are set forth in, e.g., Genbank Accession Nos. AK315310 and AAC05174.

An “agent that antagonizes leukemia inhibitory factor” or “agent that antagonizes LIF” is any agent that inhibits, inactivates, decreases, blocks, or downregulates the expression or activity of LIF. In some embodiments, an agent antagonizes LIF if it decreases the expression or activity of LIF in a biological sample (e.g., cell or tissue) contacted with the agent by at least 10%, 15%, 20%, 25%, 30%, 40%, 50%, 60%, 70%, 80%, 90% or more relative to a control sample (e.g., the biological sample prior to the contacting). In some embodiments, the agent is an anti-LIF antibody.

The term “agent” refers to any molecule, either naturally occurring or synthetic, e.g., peptide, protein, oligopeptide (e.g., from about 5 to about 25 amino acids in length, e.g., about 5, 10, 15, 20, or 25 amino acids in length), small organic molecule (e.g., an organic molecule having a molecular weight of less than about 2500 daltons, e.g., less than 2000, less than 1000, or less than 500 daltons), circular peptide, peptidomimetic, antibody, polysaccharide, lipid, fatty acid, inhibitory RNA (e.g., siRNA or shRNA), polynucleotide, oligonucleotide, aptamer, drug compound, or other compound.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab.

Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al. (1993), PNAS. USA 90:6444, Gruber et al. (1994) J Immunol.:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994).

“Single chain Fv (svFv)” or “single chain antibodes” refers to a protein wherein the V_{H} and the V_{L} regions of a scFv antibody comprise a single chain which is folded to create an antigen binding site similar to that found in two chain antibodies. Methods of making scFv antibodies have been described in e.g., Ward et al., Exp Hematol. (5):660-4 (1993); and Vaughan et al., Nat Biotechnol. 14(3):309-14 (1996). Single chain Fv (scFv) antibodies optionally include a peptide linker of no more than 50 amino acids, generally no more than 40 amino acids, preferably no more than 30 amino acids, and more preferably no more than 20 amino acids in length. In some embodiments, the peptide linker is a concatamer of the sequence Gly-Gly-Gly-Gly-Ser, e.g., 2, 3, 4, 5, or 6 such sequences. However, it is to be appreciated that some amino acid substitutions within the linker can be made. For example, a valine can be substituted for a glycine. Additional peptide linkers and their use are well-known in the art. See, e.g., Huston et al., Proc. Nat'l Acad. Sci. USA 8:5879 (1988); Bird et al., Science 242:4236 (1988); Glockshuber et al., Biochemistry 29:1362 (1990); U.S. Pat. No. 4,946,778, U.S. Pat. No. 5,132,405 and Stemmer et al., Biotechniques 14:256-265 (1993).

The phrase “specifically (or selectively) binds to an antibody”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein (e.g., LIF or a portion thereof) and do not bind in a significant amount to other proteins present in the sample. Specific binding to an antibody under such conditions may require an antibody that is selected for its specificity for a particular protein. For example, antibodies raised against LIF can be selected to obtain antibodies specifically immunoreactive with that protein and not with other proteins, except for polymorphic variants, e.g., proteins at least 80%, 85%, 90%, 95% or 99% identical to a sequence of interest. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the terms “nucleic acid” and “polynucleotide” are used interchangeably. Use of the term “polynucleotide” includes oligonucleotides (i.e., short polynucleotides). This term also refers to deoxyribonucleotides, ribonucleotides, and naturally occurring variants, and can also refer to synthetic and/or non-naturally occurring nucleic acids (i.e., comprising nucleic acid analogues or modified backbone residues or linkages), such as, for example and without limitation, phosphorothioates, phosphoramidates, methyl phosphonates, chiral-methyl phosphonates, 2-O-methyl ribonucleotides, peptide-nucleic acids (PNAs), and the like. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (see, e.g., Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

A “biological sample” includes blood and blood fractions or products (e.g., serum, plasma, platelets, red blood cells, and the like); sputum or saliva; kidney, lung, liver, heart, brain, nervous tissue, thyroid, eye, skeletal muscle, cartilage, or bone tissue; cultured cells, e.g., primary cultures, explants, and transformed cells, stem cells, stool, urine, etc. Such biological samples also include sections of tissues such as biopsy and autopsy samples, and frozen sections taken for histologic purposes. A biological sample is typically obtained from a “subject,” i.e., a eukaryotic organism, most preferably a mammal such as a primate, e.g., chimpanzee or human; cow; dog; cat; a rodent, e.g., guinea pig, rat, or mouse; rabbit; or a bird; reptile; or fish.

A “therapeutic amount” or “therapeutically effective amount” of an agent (e.g., an agent that antagonizes LIF) is an amount of the agent which prevents, alleviates, abates, or reduces the severity of symptoms of a cancer (e.g., a K-Ras-expressing cancer) in a subject.

The terms “administer,” “administered,” or “administering” refer to methods of delivering agents, compounds, or compositions to the desired site of biological action. These methods include, but are not limited to, topical delivery, parenteral delivery, intravenous delivery, intradermal delivery, intramuscular delivery, colonical delivery, rectal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

### III. Methods of Treating Cancers

In one aspect, methods for treating or preventing a cancer in a subject are provided. In some embodiments, the method comprises administering to the subject a therapeutic amount of an agent that antagonizes leukemia inhibitory factor (LIF). In some embodiments, the subject is a human, e.g., a human adult or a human child.

In some embodiments, the cancer is a K-Ras-expressing cancer, e.g., a cancer that expresses or overexpresses wild-type K-Ras or a cancer that expresses a mutated form of K-Ras. In some embodiments, the K-Ras-expressing cancer is a pancreatic cancer, a colorectal cancer, or a lung cancer. In some embodiments, the K-Ras-expressing cancer is a pancreatic cancer, e.g., pancreatic ductal adenocarcinoma. In some embodiments, the method further comprises measuring the level of K-Ras expression in a sample (e.g., a tumor tissue sample) from the subject. In some embodiments, the method further comprises determining a K-Ras genotype that is expressed in a sample (e.g., a tumor tissue sample) from the subject.

In some embodiments, the method further comprises:

detecting the level of K-Ras expression in a sample from the subject (e.g., a tumor cell or tumor tissue sample from the subject);

determining whether the level of K-Ras expression in the sample from the subject is greater than the level of K-Ras expression of a control (e.g., a non-diseased cell or tissue that does not express K-Ras, such as normal human peripheric lymphocytes); and administering an agent that antagonizes LIF to the subject when the level of K-Ras expression in the sample from the subject is greater than the level of K-Ras expression of a control.

In some embodiments, the cancer is not a K-Ras-expressing or -overexpressing cancer. As a non-limiting example, in some embodiments the cancer is a pancreatic cancer (e.g., a pancreatic ductal adenocarcinoma) that does not express or overexpress K-Ras.

K-Ras-Expressing Cancers

In some embodiments, the cancer is a cancer that expresses K-Ras at a detectable level. In some embodiments, a cancer has a detectable level of K-Ras expression when at least 0.1% of cells in the cancer tissue sample are positive for K-Ras activation (e.g., wild-type K-Ras or a K-Ras activating mutation at codon 12, codon 13, codon 61, and/or other codons). In some embodiments, the cancer has a detectable level of expression of wild-type K-Ras. In some embodiments, the cancer has a detectable level of expression of a mutated K-Ras. In some embodiments, the K-Ras mutation is an activating mutation at one or more of codon 5 (e.g., K5E), codon 9 (e.g., V9I), codon 12 (e.g., G12A, G12C, G12D, G12F, G12R, G12S, G12V, G12Y), codon 13 (e.g., G13C, G13D, G13V), codon 14 (e.g., V141, V14L), codon 18 (e.g., A18D), codon 19 (e.g., L19F), codon 22 (e.g., Q22K), codon 23 (e.g., L23R), codon 24 (e.g., I24N), codon 26 (e.g., N26K), codon 33 (e.g., D33E), codon 36 (e.g., I36L, I36M), codon 57 (e.g., D57N), codon 59 (e.g., A59E, A59G, A59T), codon 61 (e.g., Q61H, Q61K, Q61L, Q61R), codon 62 (e.g., E62G, E62K), codon 63 (e.g., E63K), codon 64 (e.g., Y64D, Y64H, Y64N), codon 68 (e.g., R68S), codon 74 (e.g., T74P), codon 92 (e.g., D92Y), codon 97 (e.g., R97I), codon 110 (e.g., P110H, P110S), codon 117 (e.g., K117N), codon 118 (e.g., C118S), codon 119 (e.g., D119N), codon 135 (e.g., R135T), codon 138 (e.g., G138V), codon 140 (e.g., P140H), codon 146 (e.g., A146T, A146V), codon 147 (e.g., K147N), codon 153 (e.g., D153N), codon 156 (e.g., F156L), codon 160 (e.g., V160A), codon 164 (e.g., R164Q), codon 171 (e.g., I171M), codon 176 (e.g., K176Q), codon 185 (e.g., C185R, C185S), and codon 188 (e.g., M188V). In some embodiments, the K-Ras mutation is a mutation at amino acid residue G12 (e.g., a G12C, G12V, G12D, G12A, G12S, G12R, or G12F substitution). In some embodiments, the K-Ras mutation is a mutation at amino acid residue G13 (e.g., a G13C or G13D substitution). In some embodiments, the K-Ras mutation is a mutation at amino acid residue Q61 (e.g., a Q61H or Q61K substitution). In some embodiments, the K-Ras mutation is a mutation at amino acid residue A146 (e.g., an A146T or A146V substitution). In some embodiments, the cancer that expresses wild-type or mutated K-Ras at a detectable level is a pancreatic cancer, a lung cancer, or a colorectal cancer.

In some embodiments, the cancer is a cancer that overexpresses K-Ras. As used herein a cancer “overexpresses” K-Ras if the level of expression of K-Ras (e.g., wild-type K-Ras or mutated K-Ras) is increased relative to a threshold value or a control sample (e.g., a non-diseased cell or tissue that does not express K-Ras, such as normal human peripheric lymphocytes, or a cancer sample from a subject known to be negative for expression of K-Ras). In some embodiments, a cancer overexpresses K-Ras if the level of expression of K-Ras (e.g., wild-type K-Ras or mutated K-Ras) is at least 10%, 20%, 30%, 40%, 50%, 75%, 100%, 150%, or 200% greater than a threshold value or the level of K-Ras expression in a control sample (e.g., a cancer known to be negative for expression of K-Ras). In some embodiments, a cancer overexpresses K-Ras if the level of expression of K-Ras (e.g., wild-type K-Ras or mutated K-Ras) is at least 2-fold, 3-fold, 4-fold, 5-fold, 6-fold, 7-fold, 8-fold, 9-fold, or more relative to a threshold value or to the level of K-Ras expression in a control sample (e.g., a cancer known to be negative for expression of K-Ras). In some embodiments, the cancer that overexpresses wild-type or mutated K-Ras is a pancreatic cancer, a lung cancer, or a colorectal cancer.

The level of expression of K-Ras in a cancer can be measured according to methods known in the art. In some embodiments, the level of K-Ras gene expression in a cancer is measured. In some embodiments, the level of K-Ras protein expression in a cancer is measured. The level of K-Ras gene or protein expression, or the detection of a K-Ras genotype, can be measured in a biological sample from a subject. In some embodiments, the biological sample comprises a cancer cell (e.g., a cell obtained or derived from a tumor). In some embodiments, the biological sample is a tumor tissue sample.

The level of K-Ras protein expression can be measured using any of a number of immunoassays known in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used (see, e.g., Self et al., Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

Specific immunological binding of an antibody to a protein (e.g., K-Ras) can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the protein marker is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. A urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously. In some embodiments, the amount of signal can be quantified using an automated high-content imaging system. High-content imaging systems are commercially available (e.g., ImageXpress, Molecular Devices Inc., Sunnyvale, Calif.).

Antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

Analysis of K-Ras nucleic acid expression levels or K-Ras genotype can be achieved using routine techniques such as Southern analysis, reverse-transcriptase polymerase chain reaction (RT-PCR), or any other methods based on hybridization to a nucleic acid sequence that is complementary to a portion of the coding sequence of interest (e.g., slot blot hybridization) are also within the scope of the present invention. Applicable PCR amplification techniques are described in, e.g., Ausubel et al. and Innis et al., supra.

General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of nucleic acid expression levels or genotype can also be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), pyrosequencing (Ronaghi et al., Science, 281:363-365 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. In some embodiments, methods for detecting nucleic acid variants include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE), and pyrosequencing.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

The analysis can be carried out in a variety of physical formats. For example, the use of microtiter plates or automation could be used to facilitate the processing of large numbers of test samples.

Alternatively, for detecting the level of protein or nucleic acid expression, antibody or nucleic acid probes can be applied to subject samples immobilized on microscope slides. The resulting antibody staining or in situ hybridization pattern can be visualized using any one of a variety of light or fluorescent microscopic methods known in the art.

Analysis of the protein or nucleic acid can also be achieved, for example, by high pressure liquid chromatography (HPLC), alone or in combination with mass spectrometry (e.g., MALDI/MS, MALDI-TOF/MS, tandem MS, etc.).

Methods of determining K-Ras genotype are described in the art. See, e.g., Kramer et al., Cell Oncol. 31:161-167 (2009); Chen et al., J. Chromatogr. A 1216:5147-5154 (2009); Lamy et al., Modern Pathology 24:1090-1100 (2011); Galbiati et al., PLoS ONE 8(3):359939 (2013); and WO 2010/048691.

Agents that Antagonize LIF

In some embodiments, a therapeutic amount of an agent that antagonizes LIF is administered to a subject in need thereof (e.g., a subject having a cancer, e.g., a K-Ras-expressing or -overexpressing cancer). In some embodiments, the agent that antagonizes LIF is a peptide, protein, oligopeptide, circular peptide, peptidomimetic, antibody, polysaccharide, lipid, fatty acid, inhibitory RNA (e.g., siRNA, miRNA, or shRNA), polynucleotide, oligonucleotide, aptamer, small organic molecule, or drug compound. The agent can be either synthetic or naturally-occurring.

Anti-LIF Antibodies

In some embodiments, the agent is an anti-LIF antibody. In some embodiments, the anti-LIF antibody is a monoclonal antibody. In some embodiments, the anti-LIF antibody is an antibody fragment such as a Fab, a F(ab′)₂, and a Fv.

In some embodiments, the anti-LIF antibody is a monoclonal antibody produced by the hybridoma cell deposited under American Type Culture Collection Accession Number ATCC HB11074 (Clone D25.1.4), ATCC HB11076 (Clone D3.14.1.), ATCC HB11077 (Clone D4.16.9), or ATCC HB11075 (Clone D62.3.2), or a humanized version thereof. Anti-LIF antibodies and methods of making anti-LIF antibodies are described in U.S. Pat. No. 5,654,157 and in WO 2011/124566, each of which is incorporated by reference herein. In some embodiments, the anti-LIF antibody is an antibody that competes with an antibody produced by the hybridoma cell deposited under American Type Culture Collection Accession Number ATCC HB11074 (Clone D25.1.4), ATCC HB11076 (Clone D3.14.1.), ATCC HB11077 (Clone D4.16.9), or ATCC HB11075 (Clone D62.3.2) for binding to an epitope. In some embodiments, the anti-LIF antibody is an antibody that binds the same epitope as an antibody produced by the hybridoma cell deposited under American Type Culture Collection Accession Number ATCC HB11074 (Clone D25.1.4), ATCC HB11076 (Clone D3.14.1.), ATCC HB11077 (Clone D4.16.9), or ATCC HB11075 (Clone D62.3.2). In some embodiments, the anti-LIF antibody is an antibody that binds to an epitope within the region comprising amino acids 160 to 202 of human LIF.

For preparing an antibody that antagonizes LIF (e.g., a recombinant or monoclonal antibody), many techniques known in the art can be used. See, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)).

The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Alternatively, phage or yeast display technology can be used to identify antibodies and heteromeric Fab fragments that specifically bind to selected antigens (see, e.g., McCafferty et al., Nature 348:552-554 (1990); Marks et al., Biotechnology 10:779-783 (1992); Lou et al. (2010) PEDS 23:311). Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can also be adapted to produce antibodies. Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; and WO 92/200373).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors. A V_{H} or V_{L} region as described herein may optionally comprise a methionine at the N-terminus.

Methods for humanizing or primatizing non-human antibodies are also known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)).

As an alternative to humanization, human antibodies can be generated. As a non-limiting example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immun., 7:33 (1993); and U.S. Pat. Nos. 5,591,669, 5,589,369, and 5,545,807.

In some embodiments, antibody fragments (such as a Fab, a Fab′, a F(ab′)₂, a scFv, or a dAB) are generated. Various techniques have been developed for the production of antibody fragments. Traditionally, these fragments were derived via proteolytic digestion of intact antibodies (see, e.g., Morimoto et al., J. Biochem. Biophys. Meth., 24:107-117 (1992); and Brennan et al., Science, 229:81 (1985)). However, these fragments can now be produced directly using recombinant host cells. For example, antibody fragments can be isolated from antibody phage libraries. Alternatively, Fab′-SH fragments can be directly recovered from E. coli cells and chemically coupled to form F(ab′)₂ fragments (see, e.g., Carter et al., BioTechnology, 10:163-167 (1992)). According to another approach, F(ab′)₂ fragments can be isolated directly from recombinant host cell culture. Other techniques for the production of antibody fragments will be apparent to those skilled in the art. In other embodiments, the antibody of choice is a single chain Fv fragment (scFv). See, e.g., PCT Publication No. WO 93/16185; and U.S. Pat. Nos. 5,571,894 and 5,587,458. The antibody fragment may also be a linear antibody as described, e.g., in U.S. Pat. No. 5,641,870.

In some embodiments, the antibody or antibody fragment can be conjugated to another molecule, e.g., polyethylene glycol (PEGylation) or serum albumin, to provide an extended half-life in vivo. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007).

An antibody or antibody fragment can be assayed for the ability to neutralize the activity of LIF. Methods of assaying inhibition of LIF activity are known in the art. As a non-limiting example, an assay can be performed to determine if the antibody or antibody fragment neutralizes the activity of LIF in a cell proliferation assay using the murine leukemic cell line DA-la. See, Moreau et al., Nature 15:690-692 (1988). Neutralizing antibodies can also be evaluated for their ability to block the binding of mLIF to LIFR in mouse pancreatic cancer cells driven by oncogenic K-Ras, for their ability to reduce pancreatic tumor formation, and/or for their ability to improve therapeutic responses of pancreatic tumors to gemcitabine in immuno-competent syngenic animal models.

Other LIF Antagonists

Additional antagonists of LIF can be readily identified according to methods well known to those of skill in the art. In some embodiments, antagonists of LIF can be identified by screening potential antagonists for the ability to compete with LIF for binding to the leukemia inhibitory factor receptor (LIFR). Competition assays are well known in the art. Typically, a competitive binding assay uses a labeled known ligand (e.g., LIF) in order to screen libraries (e.g., compound or peptide libraries) for candidates that bind to the known receptor (e.g., LIFR) with at least as much affinity as the known ligand.

In some embodiments, antagonists of LIF can be identified by screening potential antagonists for the ability to inhibit LIF bioactivity (e.g., in a cell proliferation assay). Methods of assaying for antagonists of LIF are described in the art, e.g., Fairlie et al., Biochemistry 42:13193-13201 (2003); Zhou et al., J. Endod 7:819-824 (2011).

Screening assays can be carried out in vitro, such as by using cell-based assays, or in vivo, such as by using animal models. In some embodiments, the assays are designed to screen large chemical libraries by automating the assay steps and providing compounds from any convenient source to assays, which are typically run in parallel (e.g., in microtiter formats on microtiter plates in robotic assays). The agents screened as potential antagonists of LIF can be small organic molecules, peptides, peptidomimetics, peptoids, proteins, polypeptides, glycoproteins, oligosaccharides, or polynucleotides such as inhibitory RNA (e.g., siRNA, antisense RNA).

Essentially any chemical compound can be tested for its ability to antagonize LIF. In some embodiments, the agents have a molecular weight of less than 1,500 daltons, and in some cases less than 1,000, 800, 600, 500, or 400 daltons. The relatively small size of the agents can be desirable because smaller molecules have a higher likelihood of having physiochemical properties compatible with good pharmacokinetic characteristics, including oral absorption than agents with higher molecular weight.

In some embodiments, high throughput screening methods involve providing a combinatorial library containing a large number of potential therapeutic compounds (potential LIF-antagonizing compounds). Such “combinatorial chemical or peptide libraries” can be screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

The preparation and screening of combinatorial chemical libraries is well known to those of skill in the art (see, e.g., Beeler et al., Curr Opin Chem Biol., 9:277 (2005); and Shang et al., Curr Opin Chem Biol., 9:248 (2005)). Libraries of use in the present invention can be composed of amino acid compounds, nucleic acid compounds, carbohydrates, or small organic compounds. Carbohydrate libraries have been described in, for example, Liang et al., Science, 274:1520-1522 (1996); and U.S. Pat. No. 5,593,853.

Representative amino acid compound libraries include, but are not limited to, peptide libraries (see, e.g., U.S. Pat. Nos. 5,010,175; 6,828,422; and 6,844,161; Furka, Int. J. Pept. Prot. Res., 37:487-493 (1991); Houghton et al., Nature, 354:84-88 (1991); and Eichler, Comb Chem High Throughput Screen., 8:135 (2005)), peptoids (PCT Publication No. WO 91/19735), encoded peptides (PCT Publication No. WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with β-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT Application No. PCT/US96/10287; and Vaughn et al., Nature Biotechnology, 14:309-314 (1996)), and peptidyl phosphonates (Campbell et al., J. Org. Chem., 59:658 (1994)).

Representative nucleic acid compound libraries include, but are not limited to, genomic DNA, cDNA, mRNA, inhibitory RNA (e.g., RNAi, siRNA), and antisense RNA libraries. See, e.g., Ausubel, Current Protocols in Molecular Biology, eds. 1987-2005, Wiley Interscience; and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference, or siRNA libraries, are described in, for example, Downward, Cell, 121:813 (2005) and Akashi et al., Nat. Rev. Mol. Cell Biol., 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines, and dipeptides (Hobbs et al., Proc. Nat. Acad. Sci. USA, 90:6909-6913 (1993)); analogous organic syntheses of small compound libraries (Chen et al., J. Amer. Chem. Soc., 116:2661 (1994)); oligocarbamates (Cho et al., Science, 261:1303 (1993)); benzodiazepines (e.g., U.S. Pat. No. 5,288,514; and Baum, C&EN, January 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Devices for the preparation of combinatorial libraries are commercially available. See, e.g., 357 MPS and 390 MPS from Advanced Chem. Tech (Louisville, Ky.), Symphony from Rainin Instruments (Woburn, Mass.), 433A from Applied Biosystems (Foster City, Calif.), and 9050 Plus from Millipore (Bedford, Mass.).

Agents that are initially identified as antagonizing LIF activity can be further tested to validate the apparent activity. Preferably such studies are conducted with suitable cell-based or animal models. The basic format of such methods involves administering a lead compound identified during an initial screen to an animal that serves as a model and then determining if in fact the activity of LIF is antagonized. The animal models utilized in validation studies generally are mammals of any kind. Specific examples of suitable animals include, but are not limited to, primates (e.g., chimpanzees, monkeys, and the like) and rodents (e.g., mice, rats, guinea pigs, rabbits, and the like.

Administration and Combination Therapy

The route of administration of a therapeutic agent (e.g., an agent that antagonizes LIF) can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art. In some embodiments, the agent that antagonizes LIF is administered orally, intravenously, or intraperitoneally.

In some embodiments, agent that antagonizes LIF is administered at a therapeutically effective amount or dose. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

In some embodiments, the agent that antagonizes LIF is administered in combination with a second therapeutic agent. In some embodiments, the second therapeutic agent is a chemotherapeutic agent. In some embodiments, the chemotherapeutic agent is an alkylating agent (e.g., cyclophosphamide, ifosfamide, chlorambucil, busulfan, melphalan, mechlorethamine, uramustine, thiotepa, nitrosoureas, or temozolomide), an anthracycline (e.g., doxorubicin, adriamycin, daunorubicin, epirubicin, or mitoxantrone), a cytoskeletal disruptor (e.g., paclitaxel or docetaxel), a histone deacetylase inhibitor (e.g., vorinostat or romidepsin), an inhibitor of topoisomerase (e.g., irinotecan, topotecan, amsacrine, etoposide, or teniposide), a kinase inhibitor (e.g., bortezomib, erlotinib, gefitinib, imatinib, vemurafenib, or vismodegib), a nucleoside analog or precursor analog (e.g., azacitidine, azathioprine, capecitabine, cytarabine, fluorouracil, gemcitabine, hydroxyurea, mercaptopurine, methotrexate, or thioguanine), a peptide antibiotic (e.g., actinomycin or bleomycin), a platinum-based agent (e.g., cisplatin, oxaloplatin, or carboplatin), or a plant alkaloid (e.g., vincristine, vinblastine, vinorelbine, vindesine, podophyllotoxin, paclitaxel, or docetaxel). In some embodiments, the chemotherapeutic agent is a nucleoside analog. In some embodiments, the chemotherapeutic agent is gemcitabine.

Co-administered therapeutic agents (e.g., the agent that antagonizes LIF, and a second therapeutic agent as described herein) can be administered together or separately, simultaneously or at different times. When administered, the therapeutic agents independently can be administered once, twice, three, four times daily or more or less often, as needed. In some embodiments, the administered therapeutic agents are administered once daily. In some embodiments, the administered therapeutic agents are administered at the same time or times, for instance as an admixture. In some embodiments, one or more of the therapeutic agents is administered in a sustained-release formulation.

In some embodiments, the agent that antagonizes LIF and a second therapeutic agent are administered concurrently. In some embodiments, the agent that antagonizes LIF is administered first, for example for about 1, 2, 3, 4, 5, 6, 7, 8, 9, 10, 15, 20, 25, 30, 40, 50, 60, 70, 80, 90, 100 days or more prior to administering the second therapeutic agent (e.g., chemotherapeutic agent). In some embodiments, the second therapeutic agent (e.g., chemotherapeutic agent) is administered first, for example for about 1, 2, 3, 4, 5, 6, 7, 8, 9, 10, 15, 20, 25, 30, 40, 50, 60, 70, 80, 90, 100 days or more prior to administering the agent that antagonizes LIF.

In some embodiments, the agent that antagonizes LIF (and optionally a second therapeutic agent, e.g., a chemotherapeutic agent as described herein) is administered to the subject over an extended period of time, e.g., for at least 30, 40, 50, 60, 70, 80, 90, 100, 150, 200, 250, 300, 350 day or longer.

### IV. Compositions and Kits

In another aspect, compositions and kits for use in treating or preventing a cancer (e.g., a K-Ras-expressing or -overexpressing cancer) in a subject are provided.

In some embodiments, pharmaceutical compositions comprising an agent that antagonizes LIF for use in administering to a subject having a cancer (e.g., a cancer in which wild-type K-Ras or mutated K-Ras is expressed or overexpressed) are provided. In some embodiments, the agent that antagonizes LIF (e.g., an anti-LIF antibody) is as described in Section III above. In some embodiments, a combination of an agent that antagonizes LIF and a second therapeutic agent (e.g., a chemotherapeutic agent as described herein) are formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols.

Guidance for preparing formulations for use in the present invention is found in, for example, in Remington: The Science and Practice of Pharmacy, 21^{st} Ed., 2006, supra; Martindale: The Complete Drug Reference, Sweetman, 2005, London: Pharmaceutical Press; Niazi, Handbook of Pharmaceutical Manufacturing Formulations, 2004, CRC Press; and Gibson, Pharmaceutical Preformulation and Formulation: A Practical Guide from Candidate Drug Selection to Commercial Dosage Form, 2001, Interpharm Press, which are hereby incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In some embodiments, an agent that antagonizes LIF (and optionally a second therapeutic agent, e.g., a chemotherapeutic agent as described herein) is prepared for delivery in a sustained-release, controlled release, extended-release, timed-release or delayed-release formulation, for example, in semi-permeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplary extended release formulations include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Exemplary controlled release formulations include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, an agent that antagonizes LIF (and optionally a second therapeutic agent, e.g., a chemotherapeutic agent as described herein) can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

The agent that antagonizes LIF (and optionally a second therapeutic agent, e.g., a chemotherapeutic agent as described herein) can be formulated for parenteral administration by injection, e.g., by bolus injection or continuous infusion. For injection, the compound or compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. In some embodiments, compounds can be formulated in aqueous solutions, preferably in physiologically compatible buffers such as Hanks's solution, Ringer's solution, or physiological saline buffer. Formulations for injection can be presented in unit dosage form, e.g., in ampules or in multi-dose containers, with an added preservative. The compositions can take such forms as suspensions, solutions or emulsions in oily or aqueous vehicles, and can contain formulatory agents such as suspending, stabilizing and/or dispersing agents.

The agent that antagonizes LIF (and optionally a second therapeutic agent, e.g., a chemotherapeutic agent as described herein) can be administered systemically by transmucosal or transdermal means. For transmucosal or transdermal administration, penetrants appropriate to the barrier to be permeated are used in the formulation. For topical administration, the agents are formulated into ointments, creams, salves, powders and gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplary transdermal delivery formulations include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. In some embodiments, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s).

Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

In some embodiments, kits for use in administering to a subject having a cancer (e.g., a cancer in which wild-type K-Ras or mutated K-Ras is expressed or overexpressed) are provided. In some embodiments, the kit comprises:

an agent that antagonizes leukemia inhibitory factor (LIF); and

a second therapeutic agent.

In some embodiments, the agent that antagonizes LIF (e.g., an anti-LIF antibody) is as described in Section III above. In some embodiments, the second therapeutic agent is a chemotherapeutic agent. In some embodiments, the chemotherapeutic agent is an alkylating agent, an anthracycline, a cytoskeletal disruptor, a histone deacetylase inhibitor, an inhibitor of topoisomerase, a kinase inhibitor, a nucleoside analog or precursor analog, a peptide antibiotic, a platinum-based agent, or a plant alkaloid. In some embodiments, the chemotherapeutic agent is a nucleoside analog. In some embodiments, the chemotherapeutic agent is gemcitabine.

In some embodiments, the kits can further comprise instructional materials containing directions (i.e., protocols) for the practice of the methods of this invention (e.g., instructions for using the kit for treating a cancer). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### V. Examples

The following examples are offered to illustrate, but not to limit, the claimed invention.

### Example 1: Targeting Leukemia Inhibitory Factor (LIF) to Eradicate Pancreatic Cells Expressing Oncogenic K-Ras

Activating mutations of K-Ras occur in over 90% of pancreatic cancers, but effective approaches to target oncogenic K-Ras have been difficult to develop. Thus, identification of essential factors for K-Ras-mediated malignancy may provide an alternative way to block this “undruggable” oncogene. With a high degree of sequence homology as well as common sets of downstream effectors and upstream affecters, the three isoforms of Ras, N-, H- and K-Ras, have long been assumed to be functionally redundant. However, K-Ras, not N- or H-Ras, deficiency in mice leads to embryonic lethality, suggesting K-Ras may be required for the functions of stem cells (Koera et al., Oncogene 15:1151-1159 (1997)). Cancer stem cells (CSCs), sharing certain similar gene expressing signatures and biological functions with normal stem cells, have been identified in numbers of human malignancies, including pancreatic adenocarcinoma (PADC) (Sampieri and Fodde, Semin Cancer Biol 22:187-193 (2012)). Due to their self-renewal, tumor initiation, chemo-resistance, and metastatic properties, CSCs are postulated to underlie treatment failures. Despite the putative role of K-Ras activation in pancreatic carcinogenesis, the roles of oncogenic K-Ras in pancreatic CSCs have not been convincingly demonstrated. In a preliminary study, we found that oncogenic K-Ras, unlike H-Ras, causes CSC-like properties in transformed mouse fibroblast and pancreatic cancer cells (data not shown).

The signal transducer and activator of transcription 3 (STAT3) is a crucial regulator of stem cell self-renewal, cancer cell survival, and metastasis. The majority of PDAC show constitutive activation of STAT3. In the Pdx1-Cre; LSL-KRAS^{G12D} transgenic mice model, loss of STAT3 reduced pancreatic tumor formation and progression, suggesting its potential as a therapeutic target in oncogenic K-Ras-induced pancreatic cancers (Corcoran et al., Cancer Res 71:5020-5029 (2011)). However, most of the STAT3 inhibitors reported to date have presented limited clinical efficacy. Therefore, alternative approaches are needed to inhibit STAT3 as potential anti-pancreatic cancer therapy.

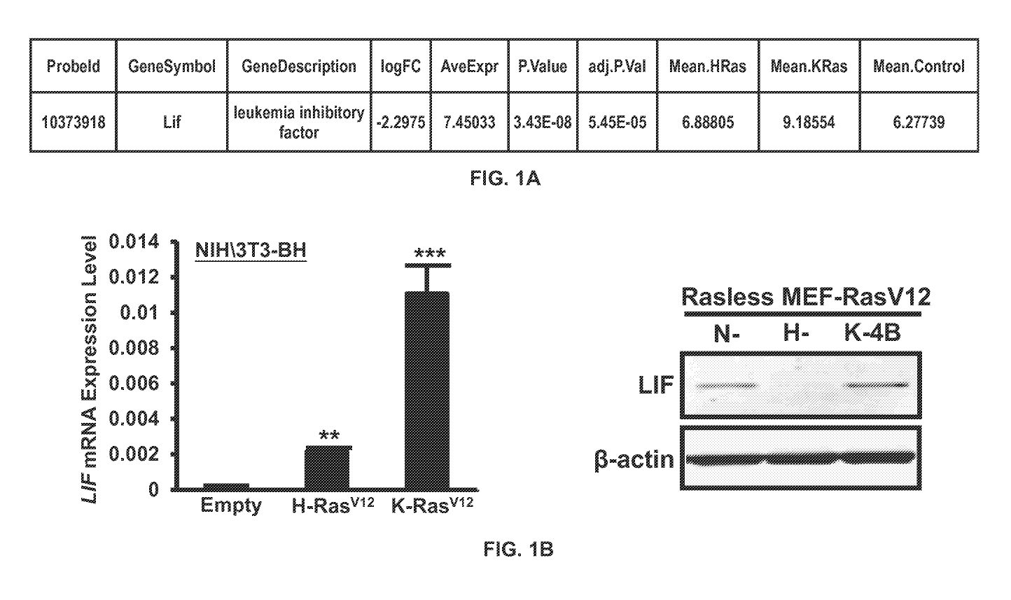

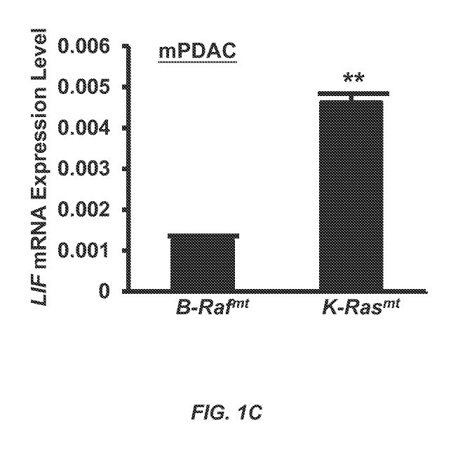

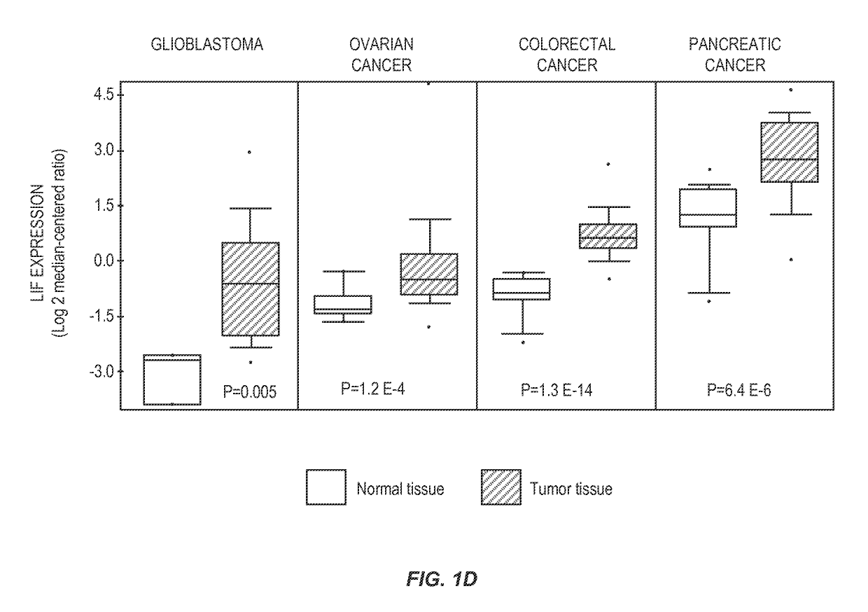

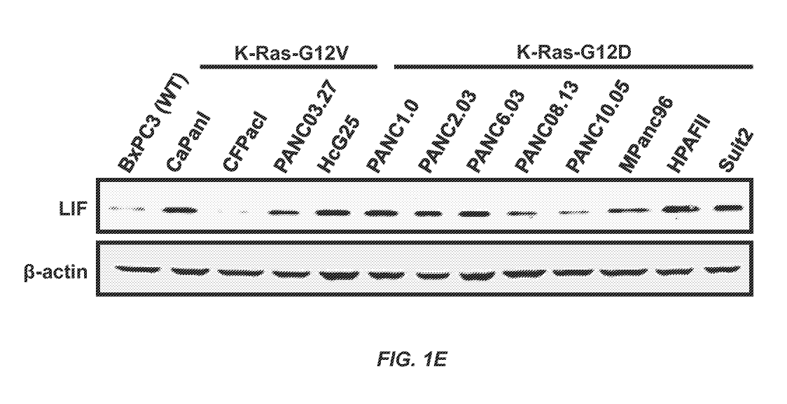

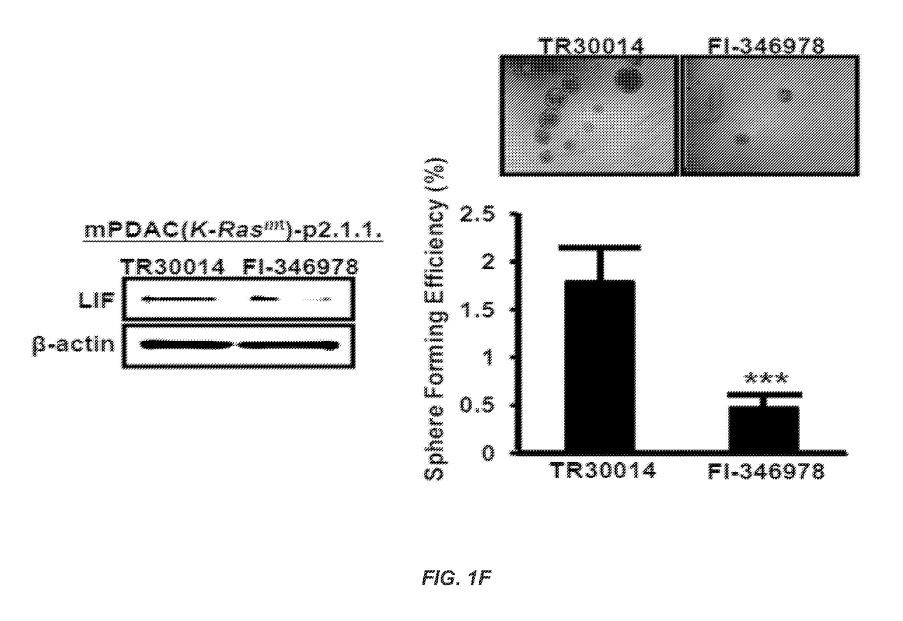

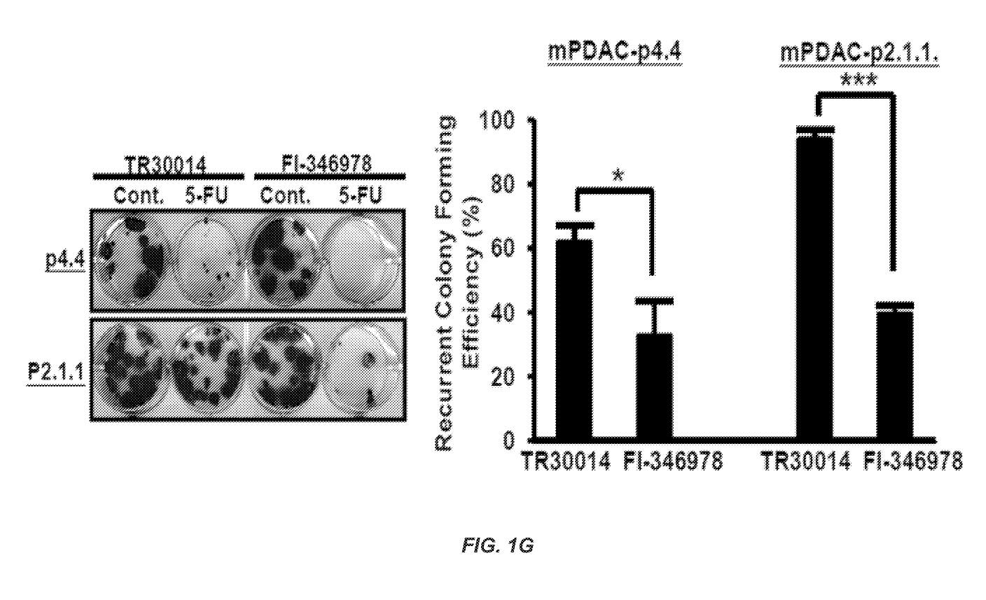

Through genome wide expression analysis in Ras^{V12}-transformed NIH3T3 cells, we identified leukemia inhibitory factor (LIF), a stem cell regulatory chemokine and STAT3 activator, as a factor markedly unregulated by K-Ras, but not by H-Ras (FIG. 1A). Moreover, oncogenic K-Ras induced mouse PDACs showed enhanced LIF expression compared to those induced by oncogenic B-Raf (FIG. 1C). Further studies demonstrated LIF is required in K-Ras-mediated sternness, including sphere formation ability and drug-resistance, in pancreatic cancer cells (FIG. 1F-1G). In a syngenic animal model, mice with orthotropic transplantation of K-Ras-driven mPDACs which LIF had been knock-down showed greater probability of survival, and impaired spleen metastasis (FIG. 1H).

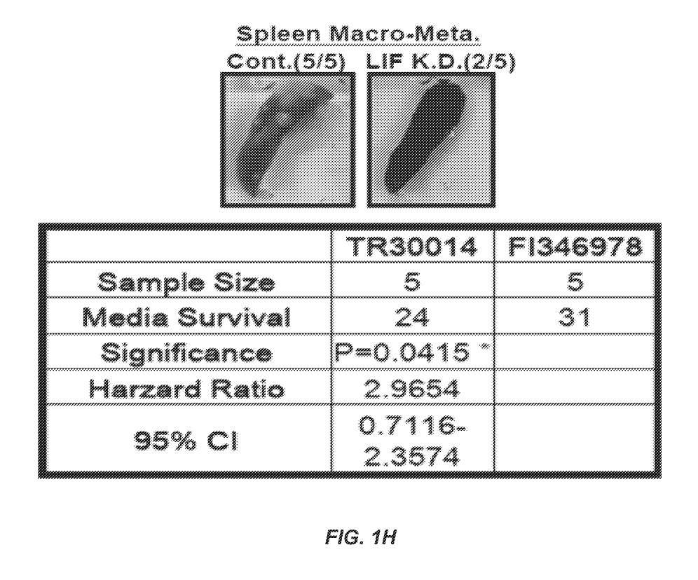

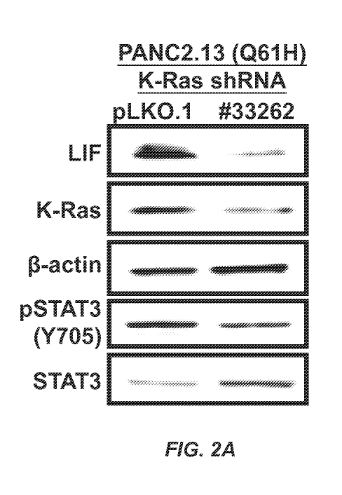

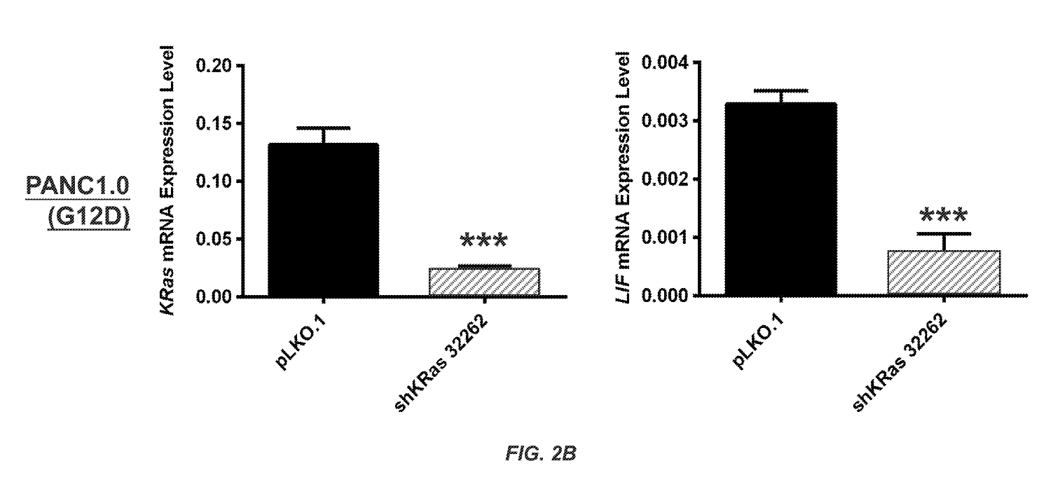

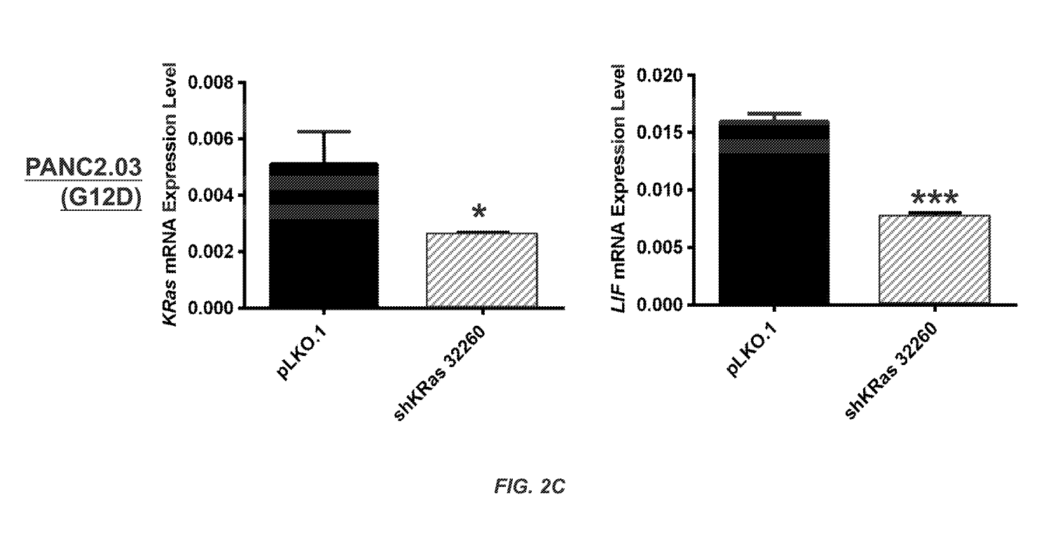

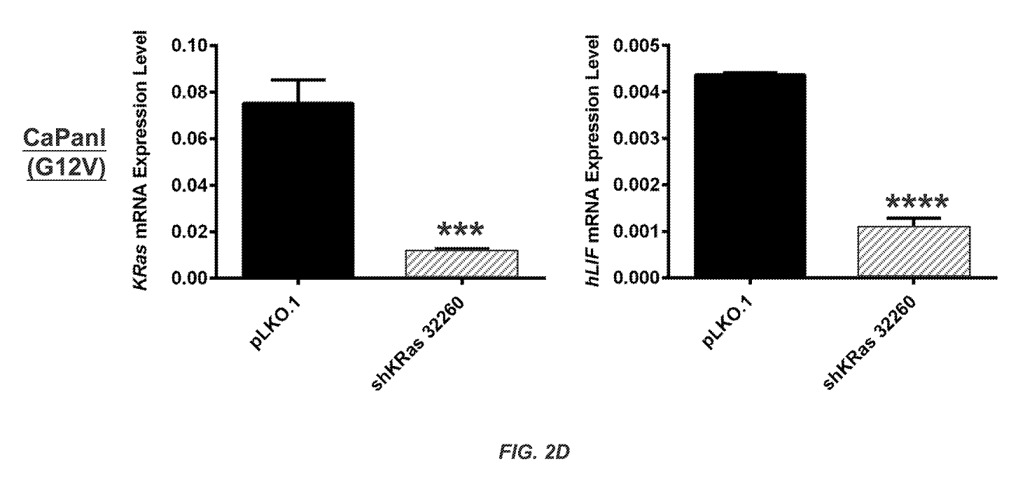

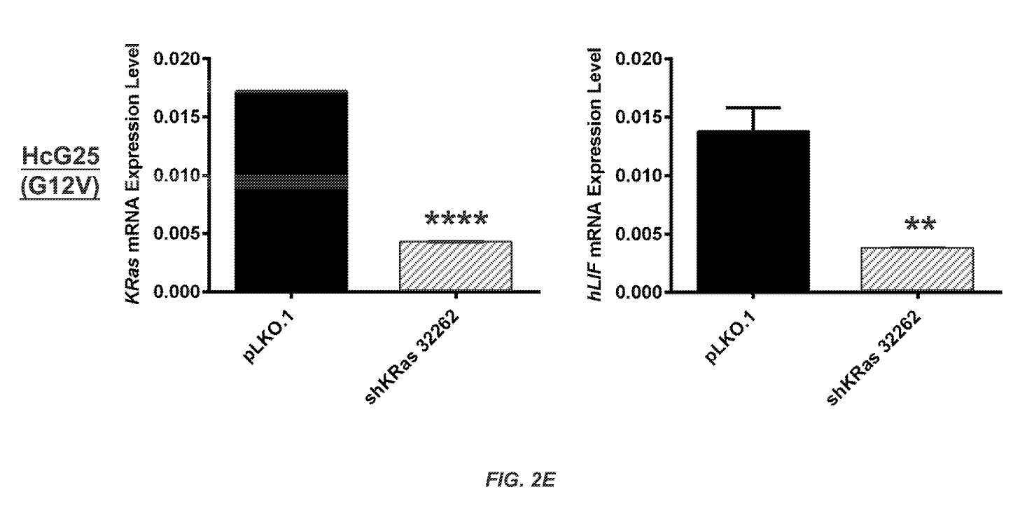

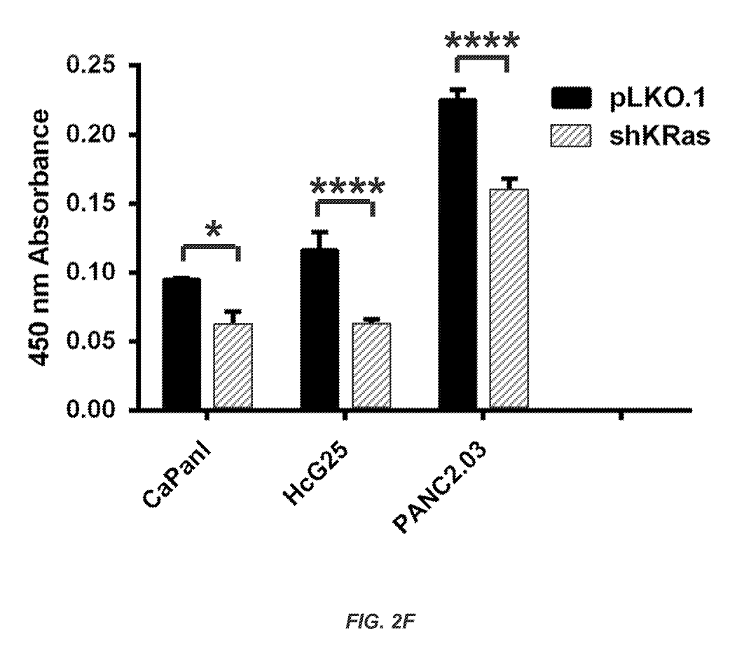

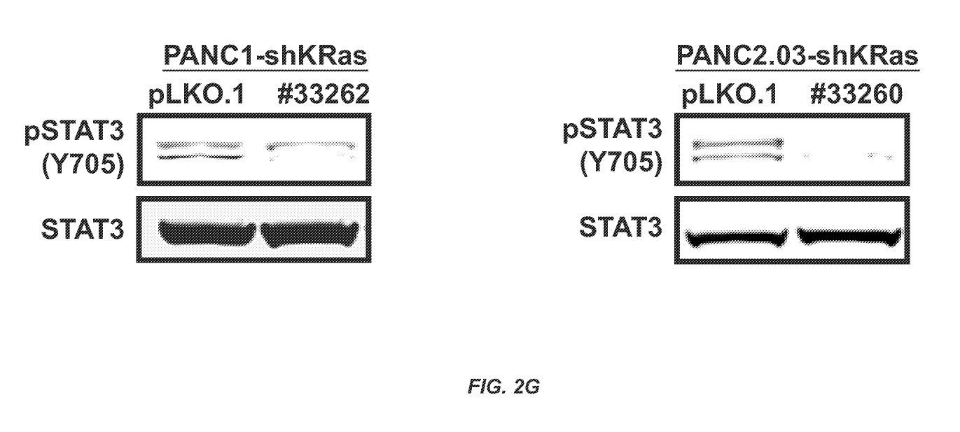

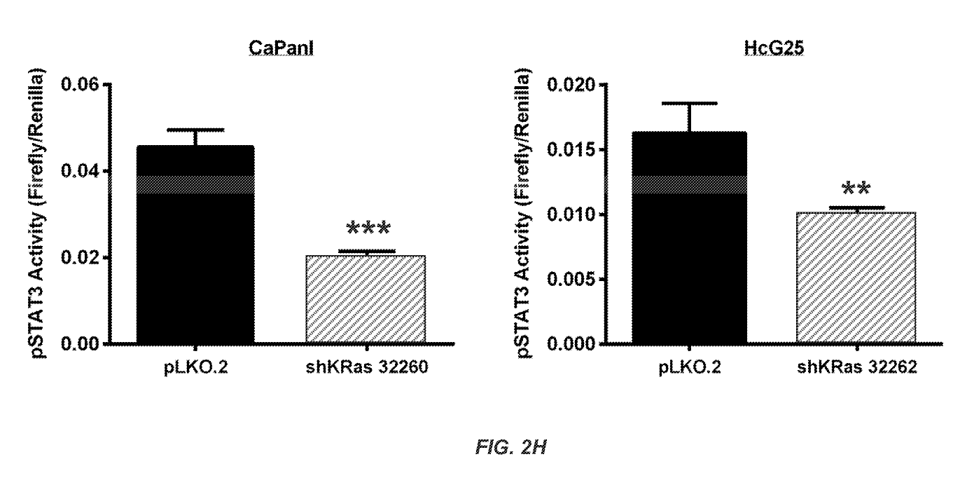

Based on these preliminary data, we hypothesized that LIF plays an essential role in the stemness of pancreatic cancer cells with activated K-Ras. Therefore, LIF represents a novel therapeutic target to eradicate K-Ras-driven pancreatic cancers. To test this hypothesis, we validated LIF as a therapeutic target of K-Ras driven pancreatic cancer by knocking down LIF via small hairpin RNA in mouse pancreatic cancer cells driven by mutant K-Ras (FVB background; LSC-K-Ras^{G12D}; p53^{F/+}, pDX^{CRE}) Knock-down efficiency was confirmed by quantitative PCR and western blotting analysis. Knock-down of K-Ras by shRNA repressed LIF protein expression in multiple human pancreatic cancer lines (FIG. 2A-2E). Additionally, a LIF ELISA revealed that human pancreatic cancer cell lines with knock-down expression of K-Ras secreted significantly decreased LIF in culture media in the comparison to control cells (FIG. 2F).

Levels and phosphorylation of STAT3 were examined by Western blot and using a STAT3 responsive luciferase assay. Western blot analysis and phospho-STAT luciferase reporter assays indicated that the pancreatic cancer cells, with knock-down expression of K-Ras and sequentially down-regulated expression of LIF, had decreased phospho-STAT3 levels (FIG. 2G) and significantly decreased STAT3 transcriptional activity (FIG. 2H).

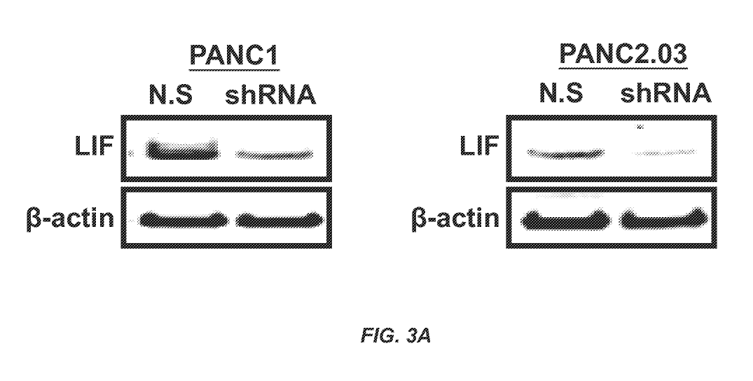

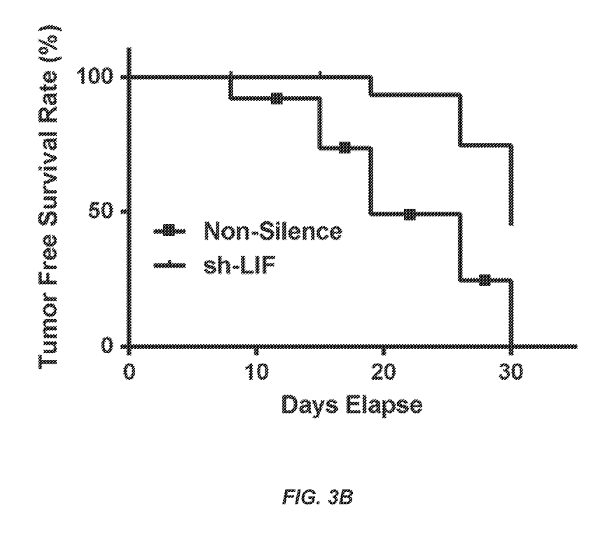

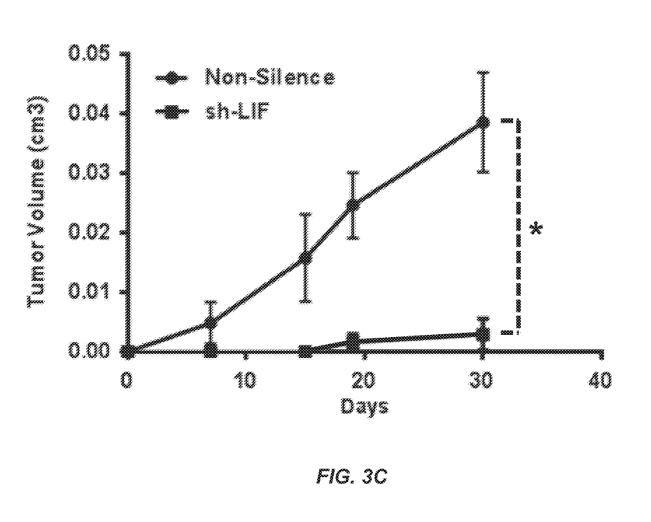

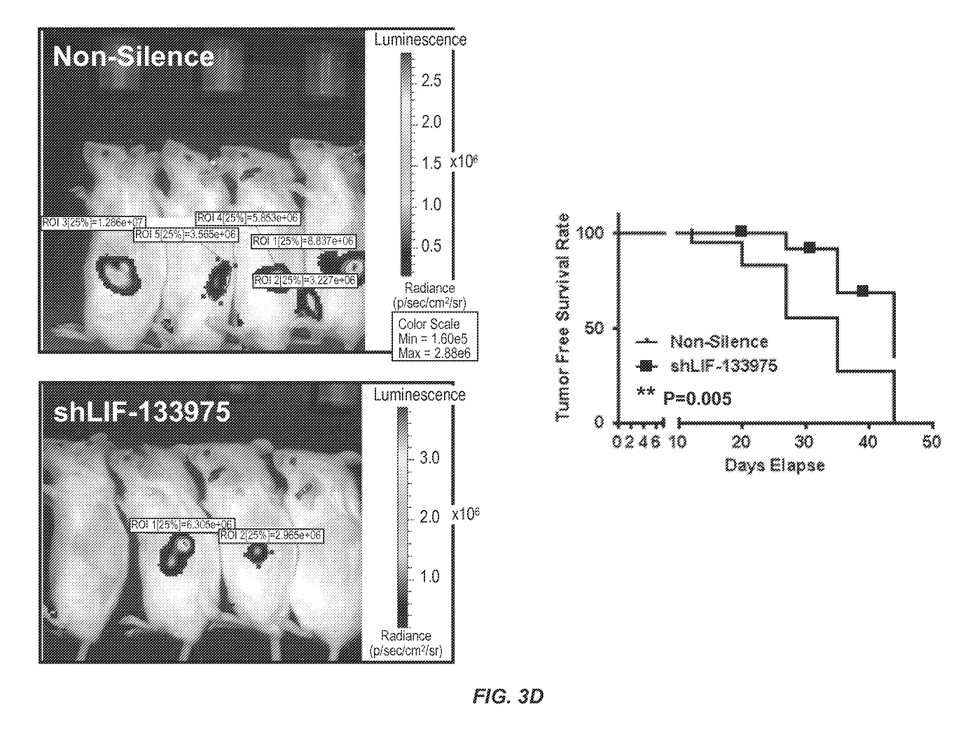

For further validation that LIF-LIFR signaling acts as a potential clinical therapeutic target, LIF was also knocked-down in human pancreatic cancer cell lines with active K-Ras mutation via small hairpin RNA (FIG. 3A). As shown in FIG. 3B, a tumor-free survival curve of PANC2.03 in subcutaneous xenograft model suggested that the cancer cells with knock-down expression of LIF possessed dramatically reduced tumor-initiating ability, when compared to control cells. Additionally, the pancreatic tumors in subcutaneous xenograft with knock-down expression of LIF grew at a significantly slower rate when compared to control tumors (FIG. 3C). Additionally, knock-down of LIF expression reduced tumor initiation rate in the PANC1-driven tumors in an orthotopic model (FIG. 3D).

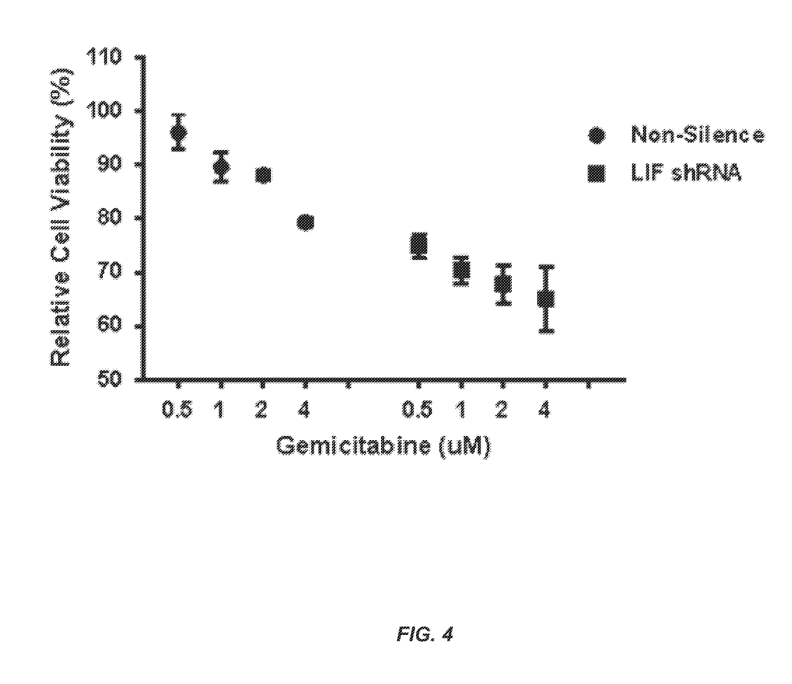

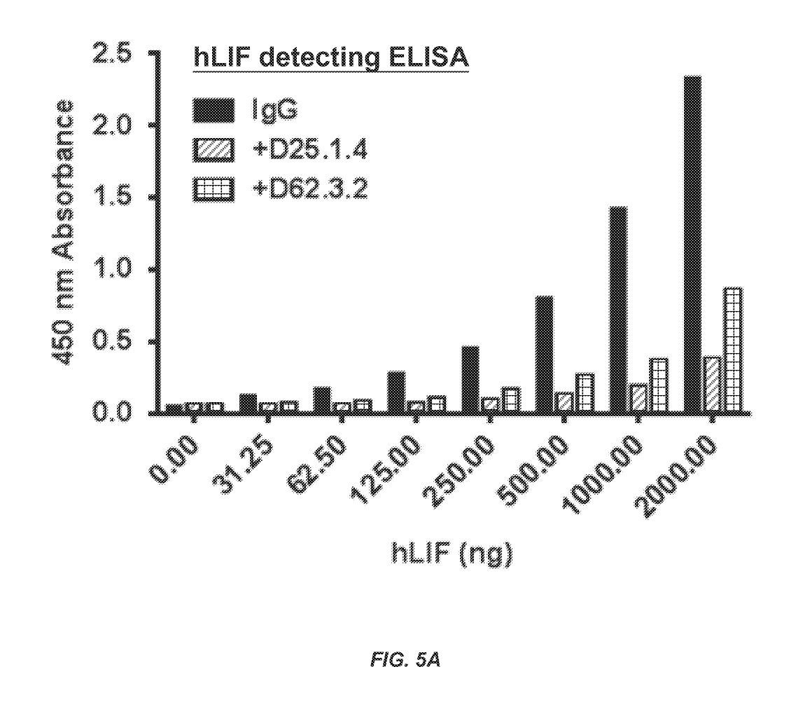

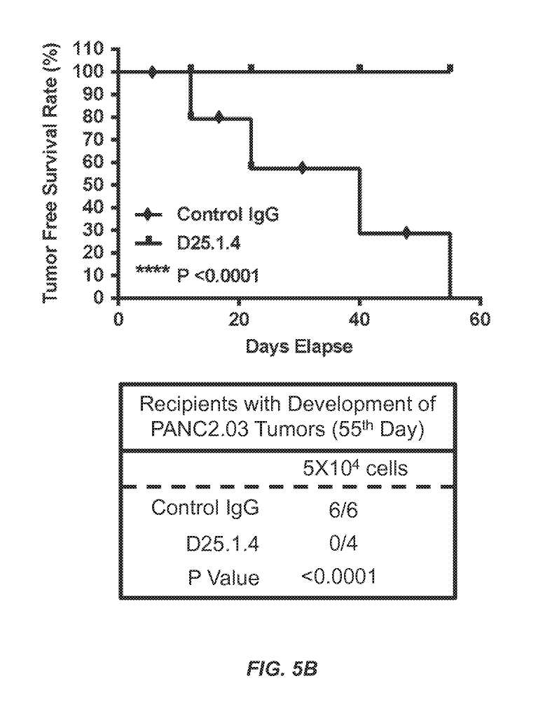

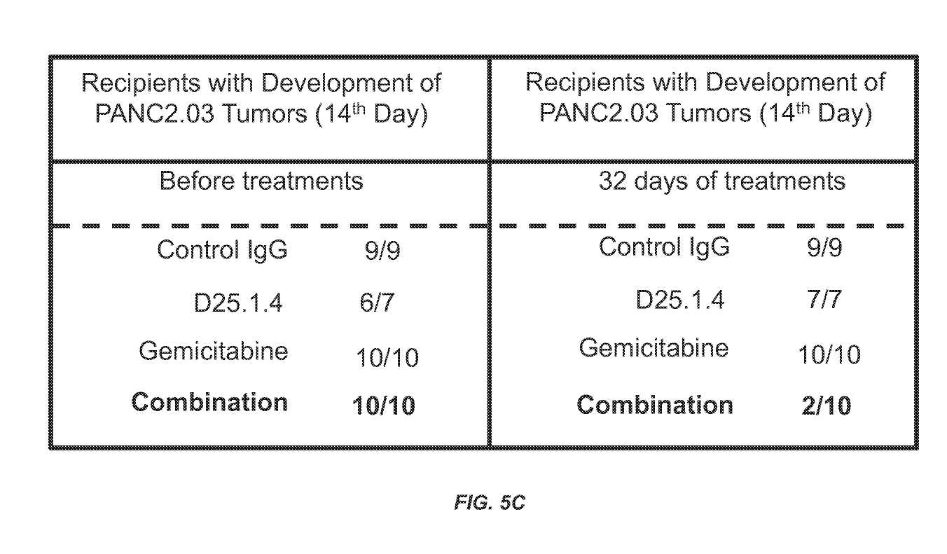

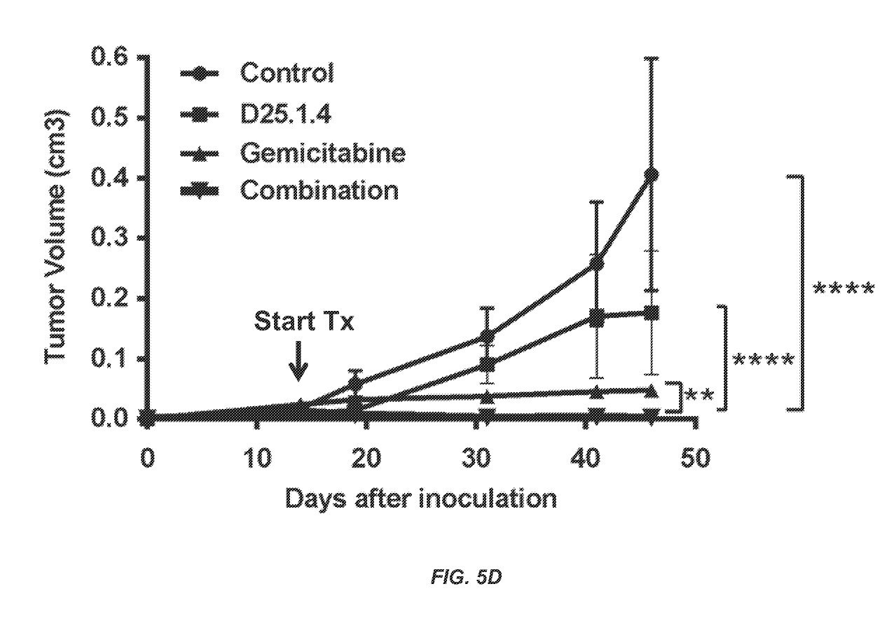

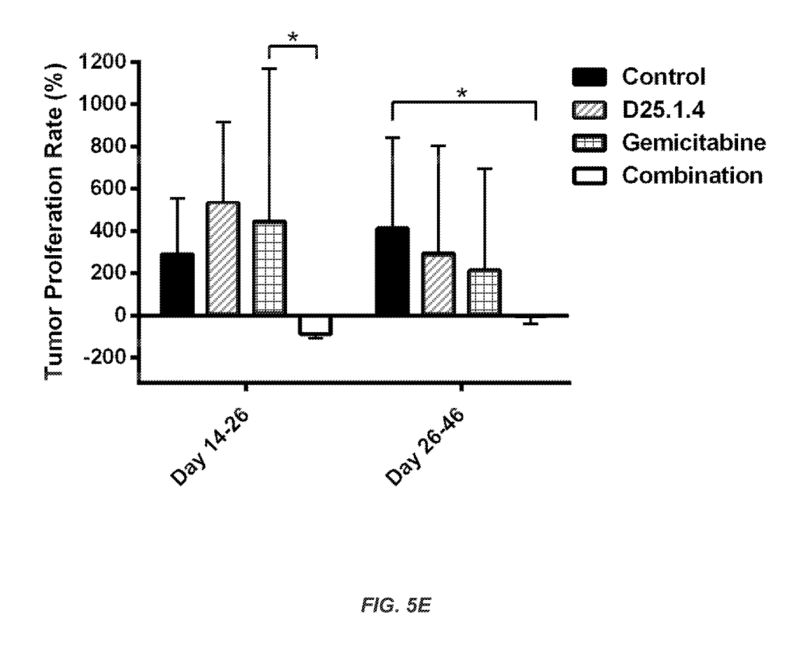

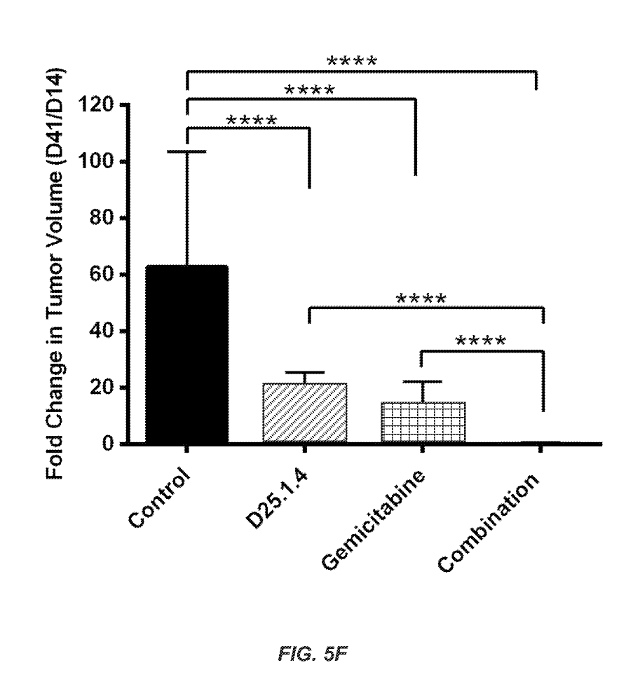

The response to gemcitabine was analyzed in pancreatic cancer cells. As shown in FIG. 4, an MTS assay suggested that knock-down of LIF sensitized PANC2.03 cells to gemcitabine treatment when compared to control cells. The effects of LIF-neutralizing antibody were also tested in mouse models. As shown in FIG. 5B, anti-LIF antibodies dramatically prevented tumor initiation when injected into nude mice prior to tumor inoculation. In a drug sensitization assay, PANC2.03 cells were subcutaneously injected into nude mice to form tumors, then the mice were treated with anti-LIF antibody, gemcitabine, or a combination of anti-LIF antibody and gemcitabine. The combination treatment of gemcitabine and anti-LIF antibody caused complete regression in 8 out of 10 tumors, whereas anti-LIF antibody alone or gemcitabine alone did not lead to the tumor regression (FIG. 5C). Additionally, the combination treatment of gemcitabine and anti-LIF antibody dramatically reduced the tumor proliferation rate, whereas the tumors treated with gemcitabine alone still had a positive growth rate (FIG. 5E-5F).

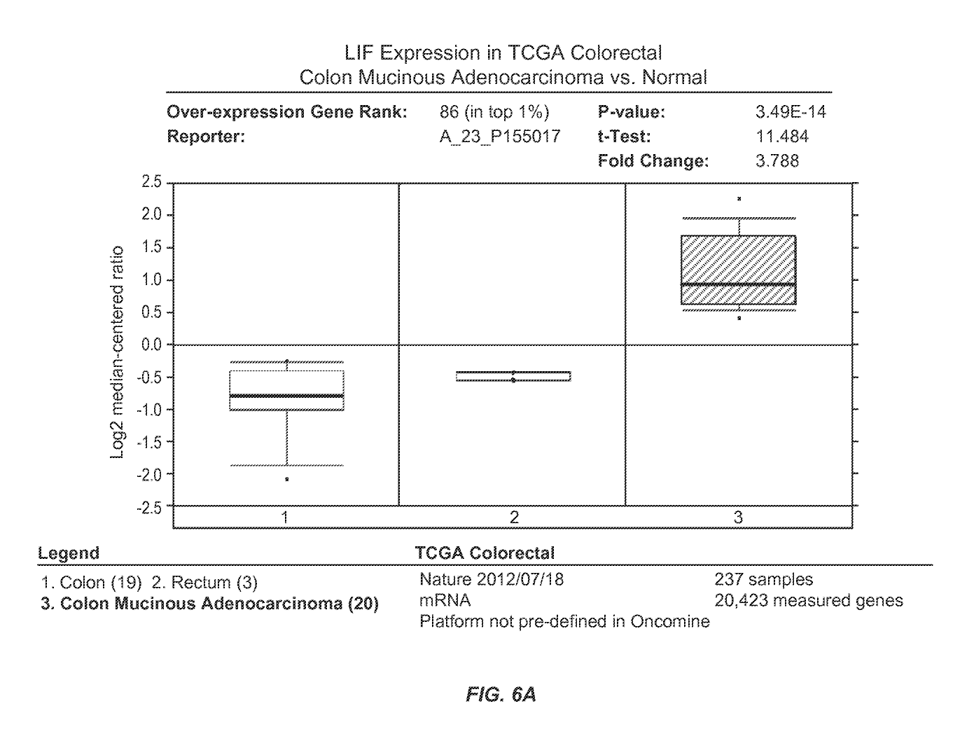

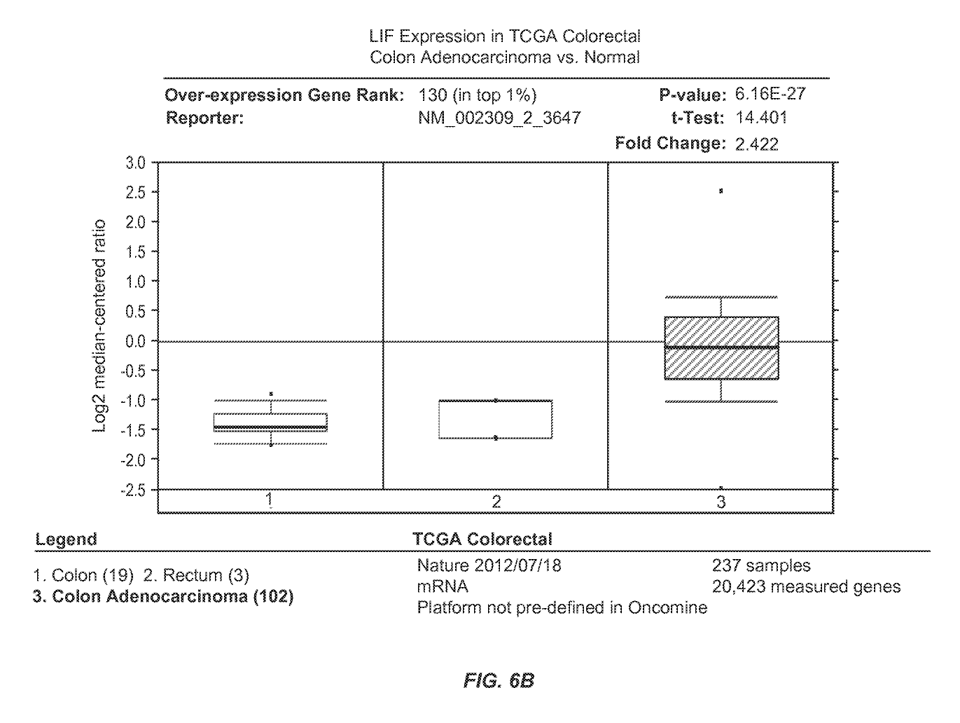

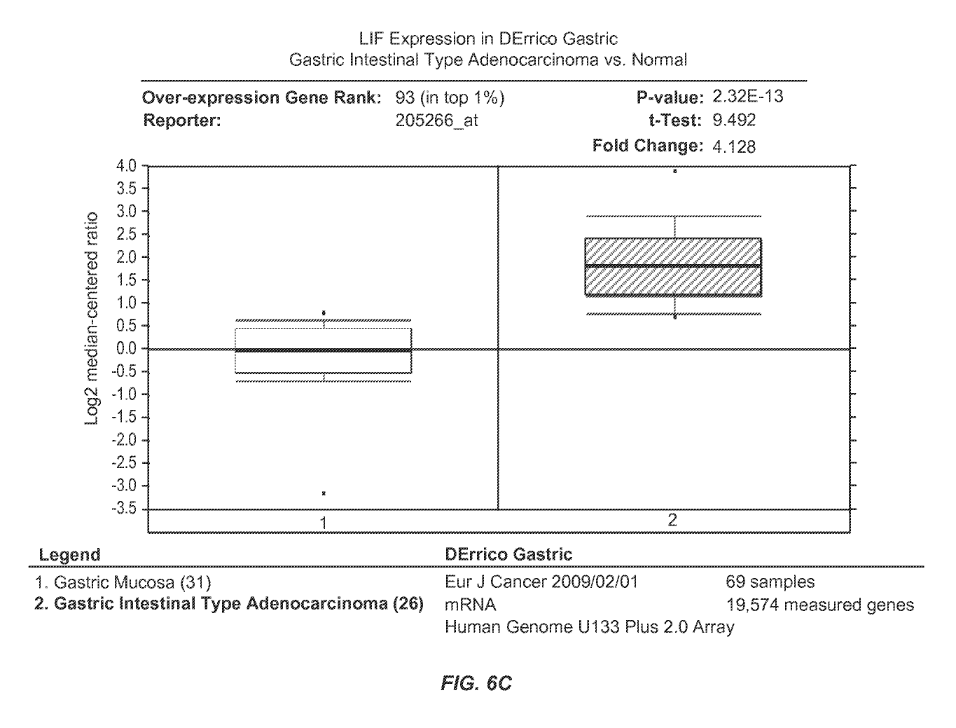

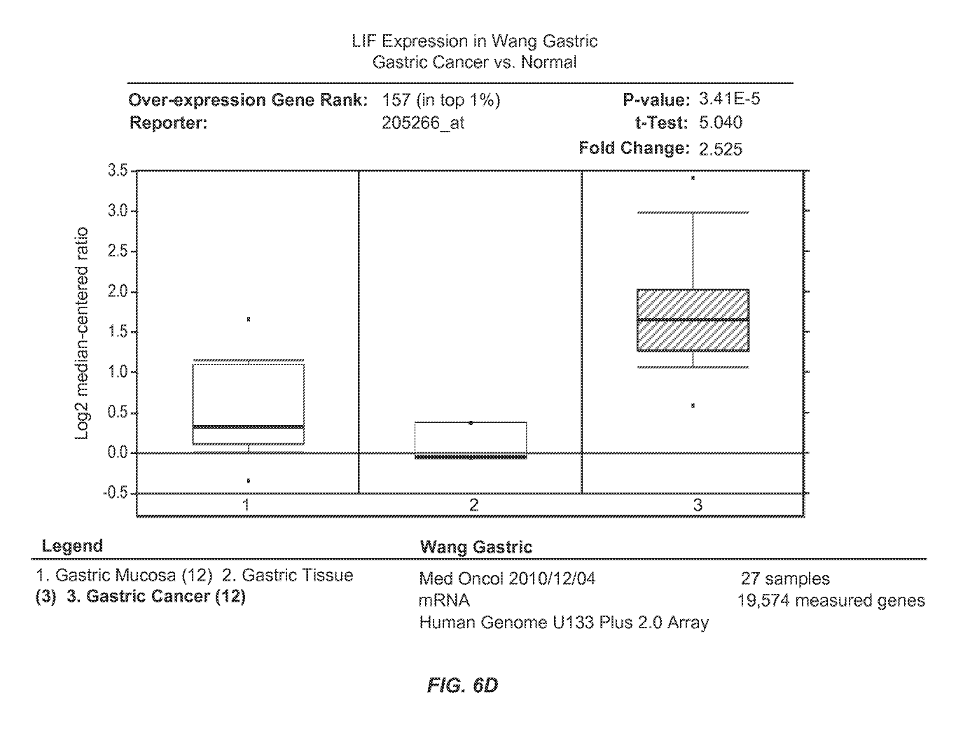

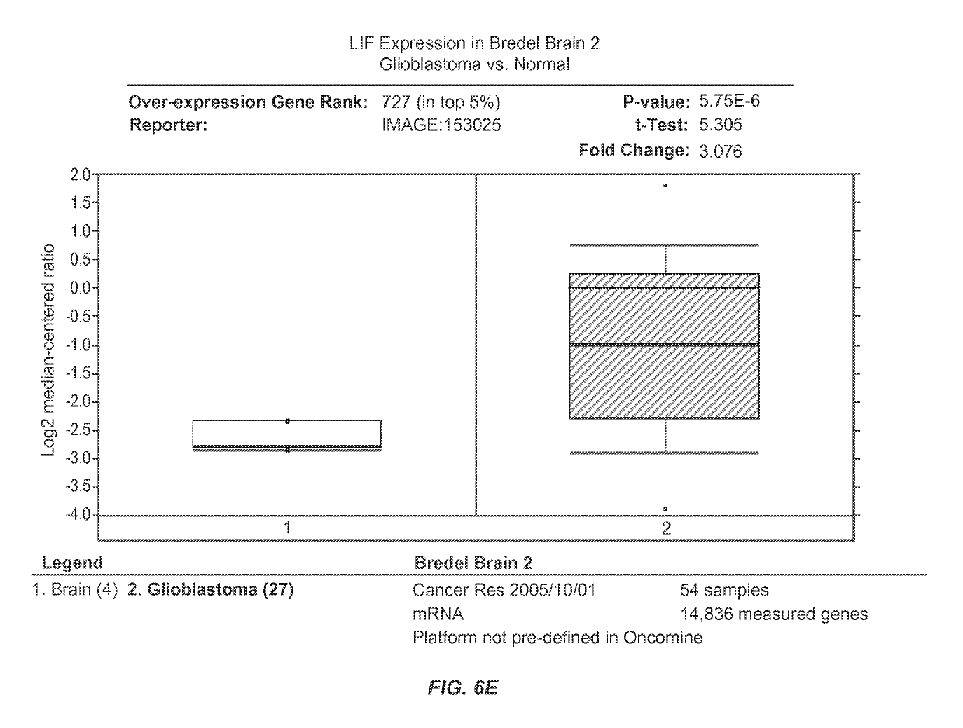

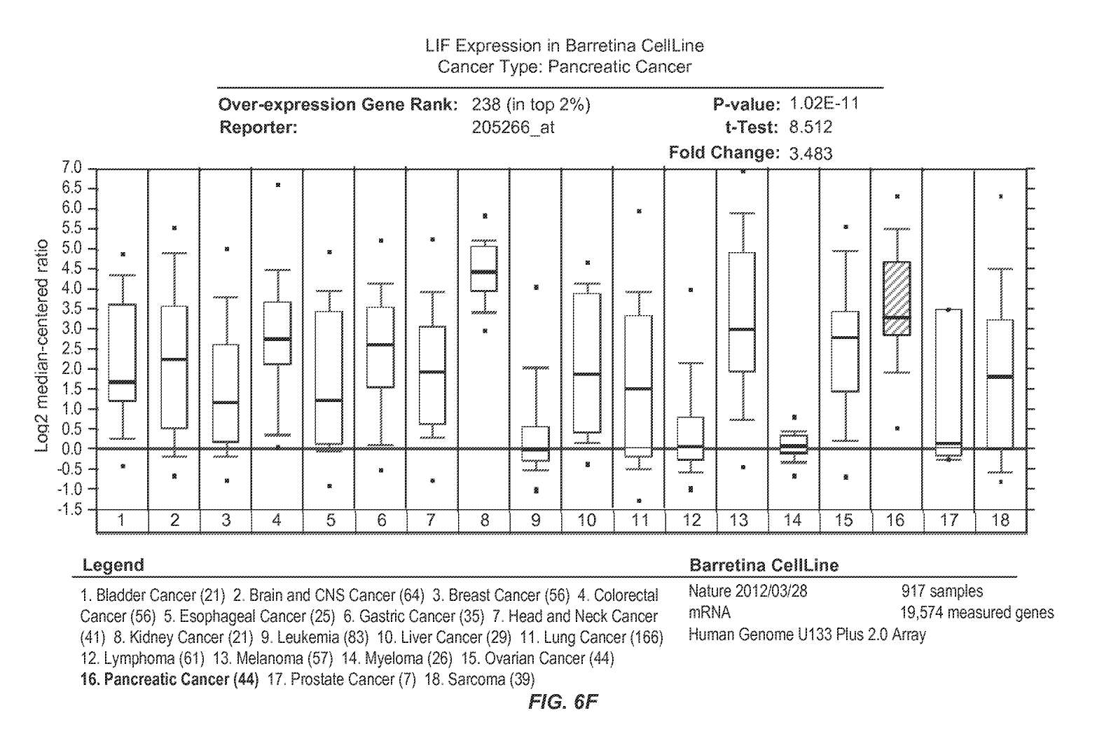

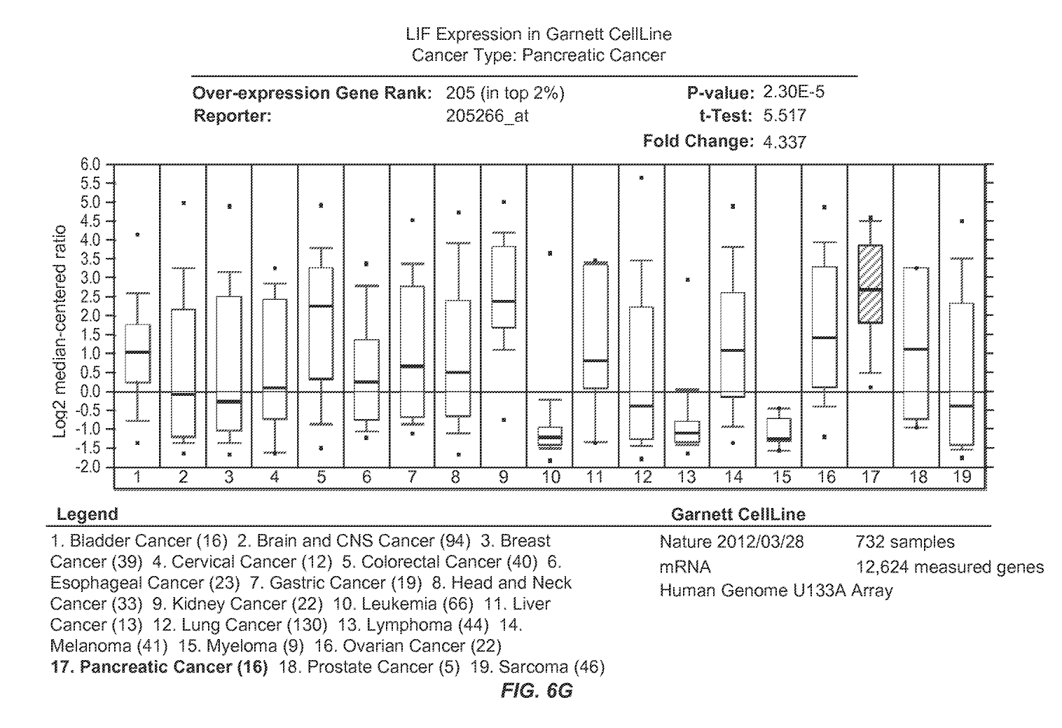

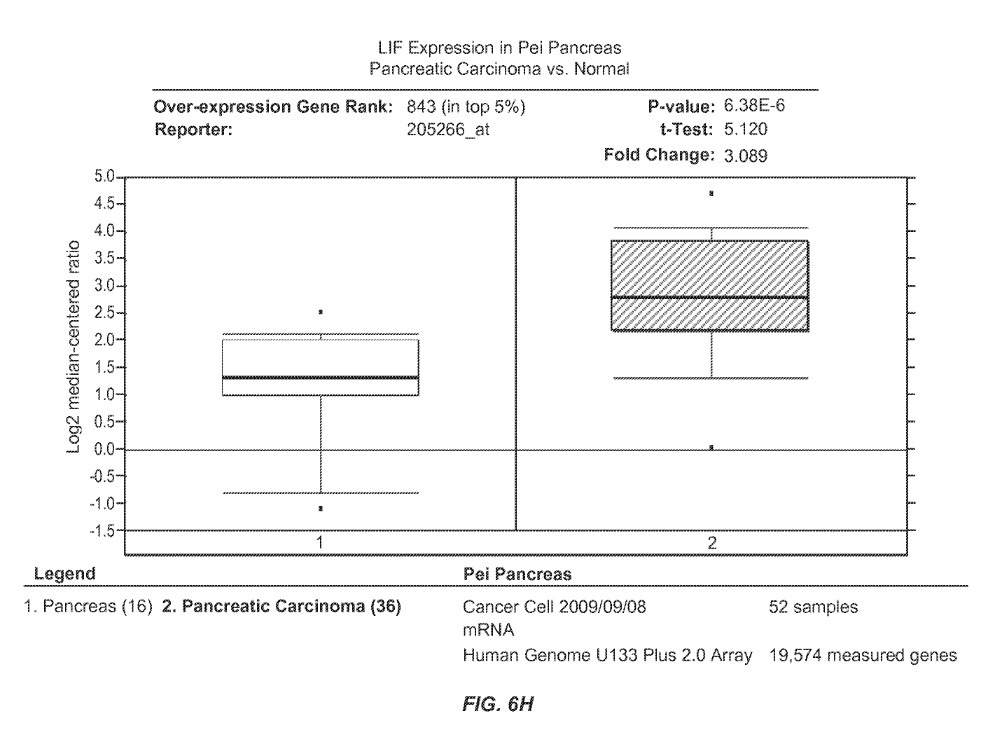

We used the online software, Oncomine™ (Invitrogen), to analyze different published datasets (as indicated in FIG. 6A-I). Oncomine™ is an online database, collecting large published genome-wild microarray data. The collected data are examined and published by other research groups. Interestingly, we found that human LIF was overexpressed in different types of cancers, including colon and pancreatic cancers, across multiple microarray platforms and published databases. Furthermore, as shown in FIG. 61, LIF expression at mRNA was significantly enhanced in the established cancer cell lines with mutant K-Ras when compared to the ones with wild type K-Ras expression. These results suggest the potential role of LIF as an oncogene in different types of human cancers, especially K-Ras driven tumors.

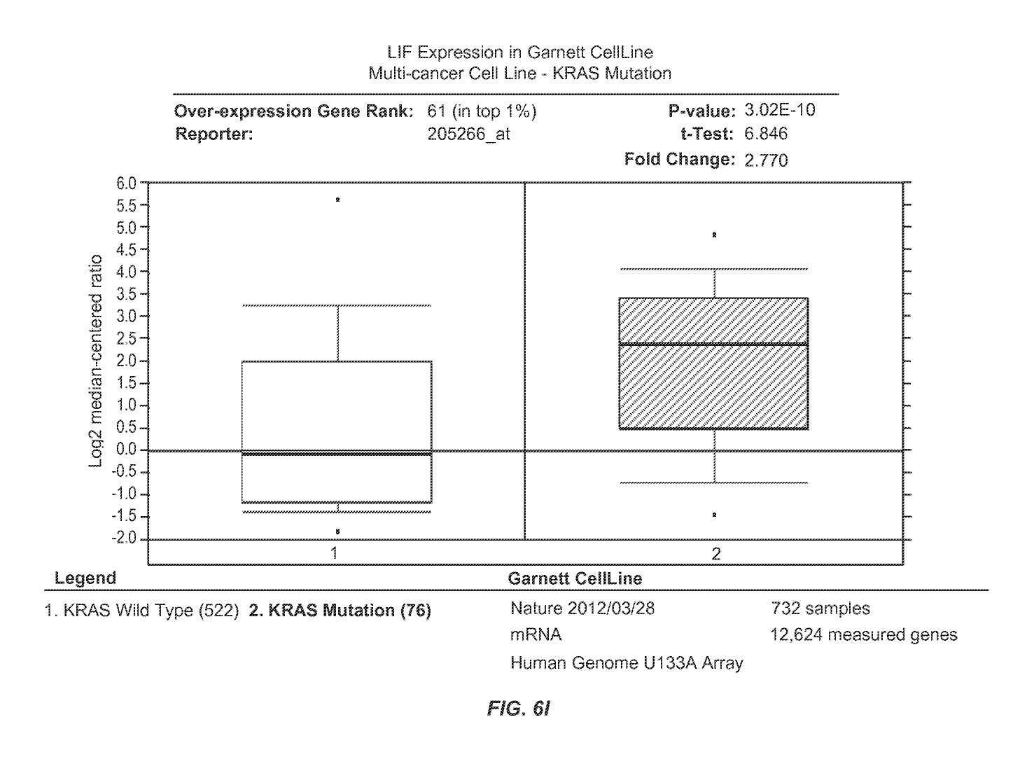

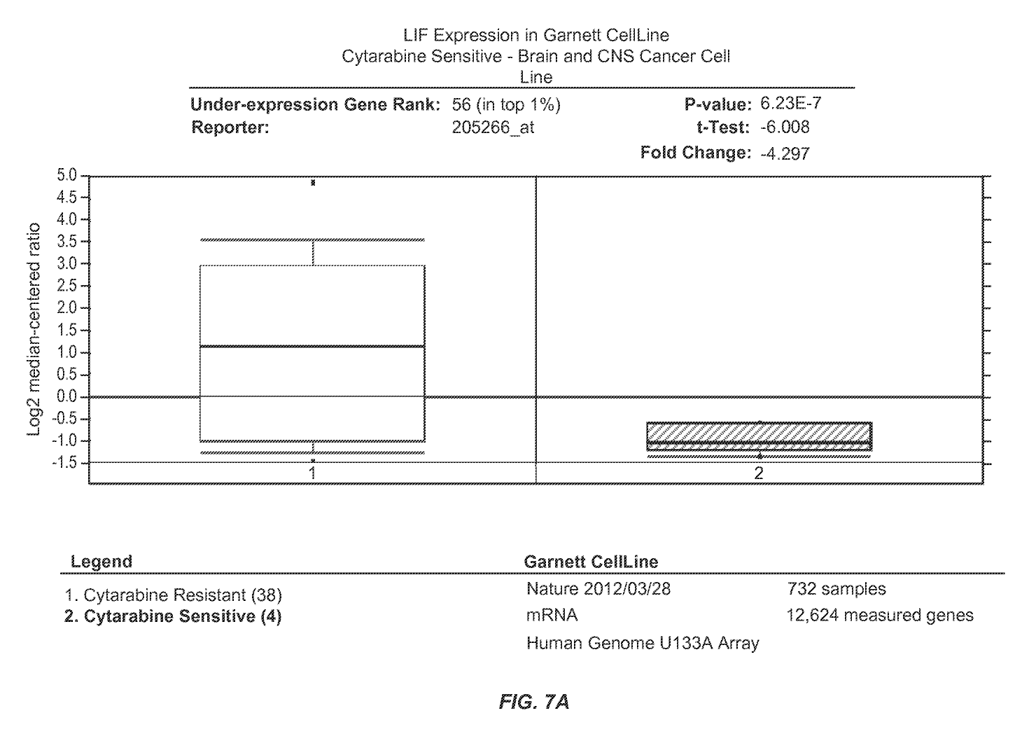

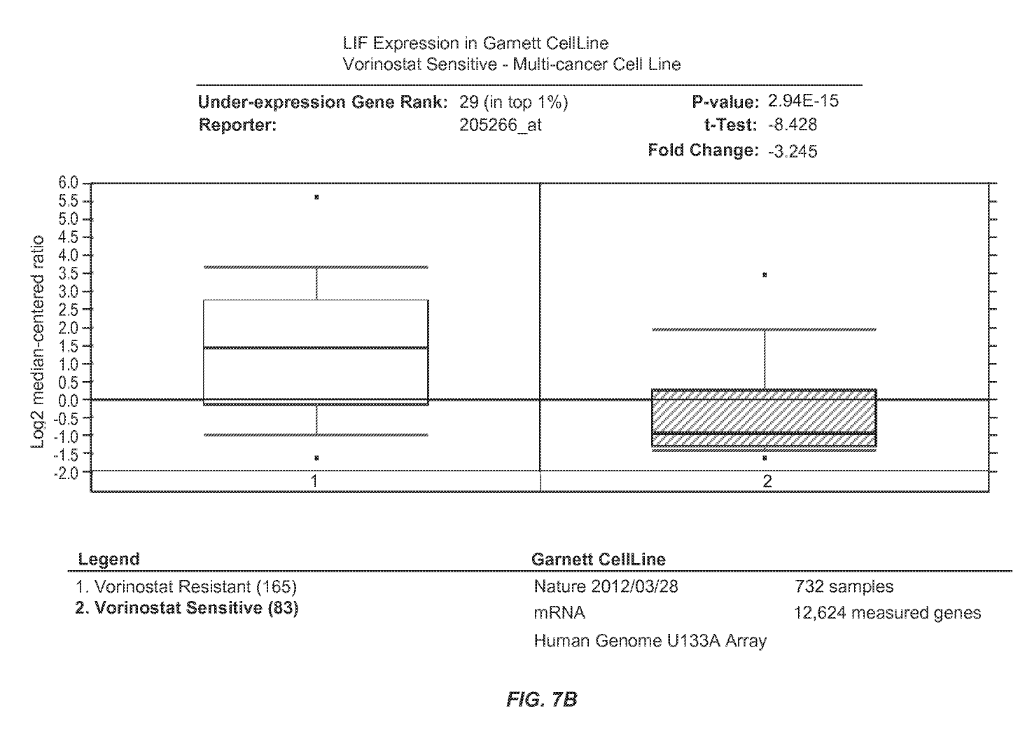

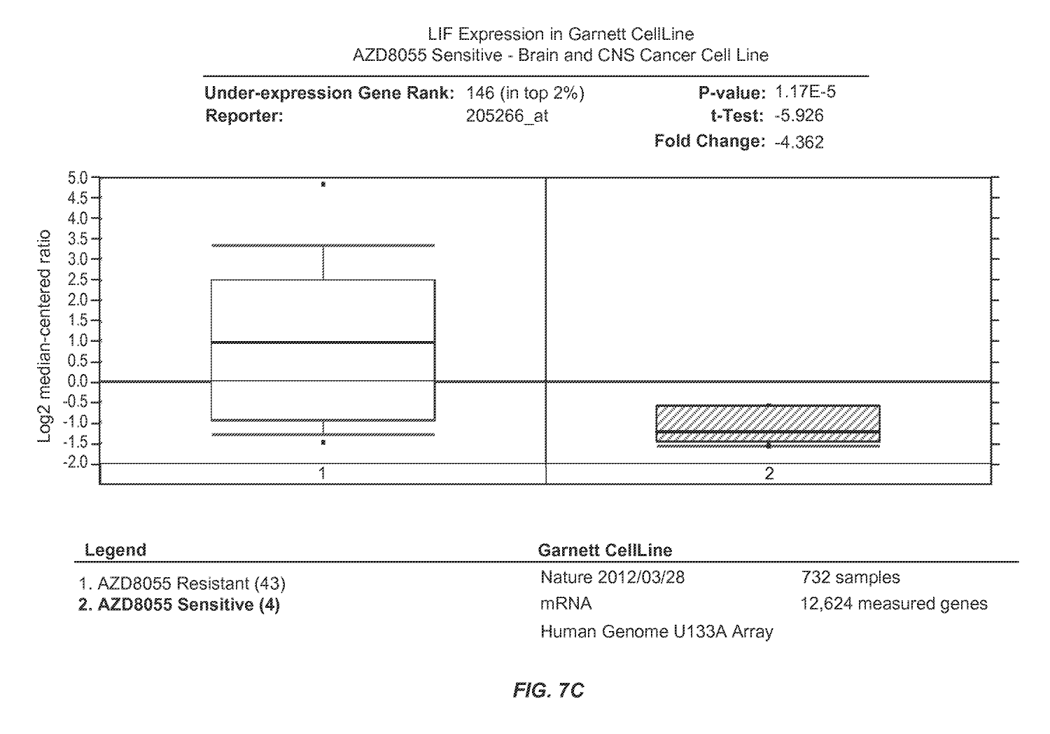

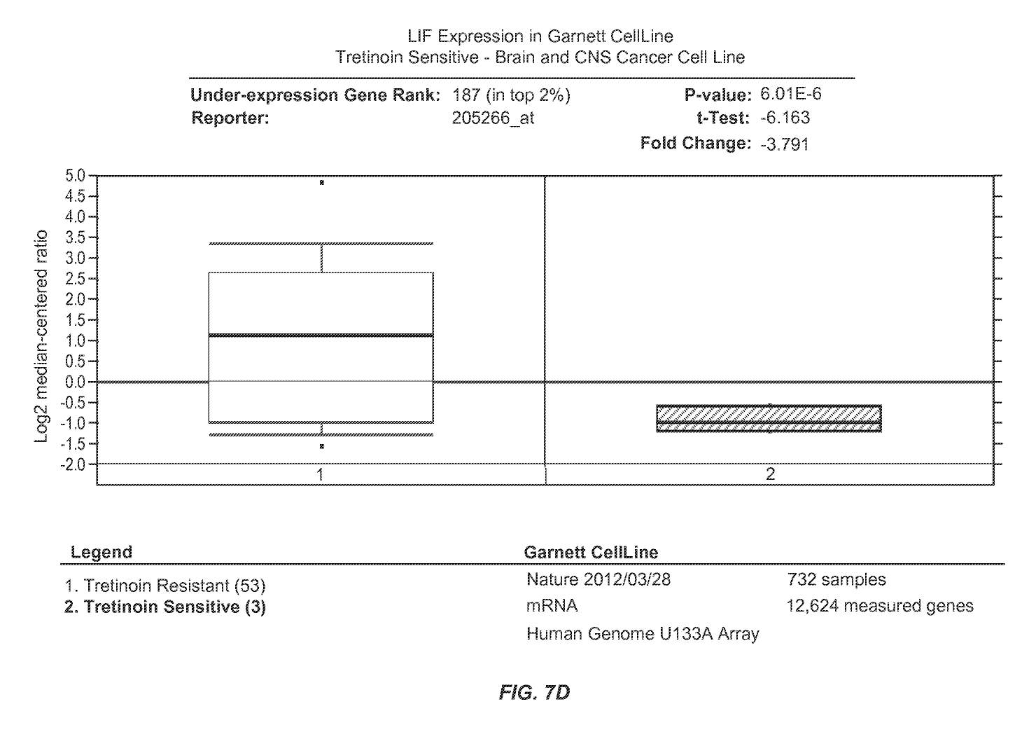

We also used Oncomine™ to analyze LIF expression at mRNA in chemotherapy-sensitive and chemotherapy-resistant cancers. Oncomine™ provides some datasets with gene profiles of chemotherapy-sensitive and chemotherapy-resistant tumor specimens. Interestingly, as shown in FIG. 7A-D, LIF expression was much higher in the tumors that were resistant to different chemotherapy treatments when compared to chemotherapy-sensitive tumors, suggesting that LIF may play an important role in the resistance of human cancers toward chemotherapies. Therefore, targeting LIF with a neutralizing antibody may re-sensitize tumor cells to conventional chemotherapy treatments.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of treating a K-Ras-expressing cancer in a subject, the method comprising administering to the subject a therapeutic amount of an agent that antagonizes leukemia inhibitory factor (LIF).

2. The method of claim 1, wherein the K-Ras-expressing cancer is a pancreatic cancer, a colorectal cancer, or a lung cancer.

3. The method of claim 1 or 2, wherein the K-Ras-expressing cancer is a pancreatic cancer.

4. The method of any of claims 1-3, wherein the pancreatic cancer is a pancreatic ductal adenocarcinoma.

5. The method of any of claims 1-4, wherein the agent that antagonizes LIF is an anti-LIF antibody.

6. The method of claim 5, wherein the anti-LIF antibody is a monoclonal antibody.

7. The method of claim 5, wherein the anti-LIF antibody is an antibody fragment selected from the group consisting of a Fab, a F(ab′)₂, and a Fv.

8. The method of any of claims 1-7, wherein the agent that antagonizes LIF is administered orally, intravenously, or intraperitoneally.

9. The method of any of claims 1-8, wherein the agent that antagonizes LIF is administered in combination with a chemotherapeutic agent.

10. The method of claim 9, wherein the chemotherapeutic agent is gemcitabine.

11. The method of claim 9 or 10, wherein the agent that antagonizes LIF and the chemotherapeutic agent are administered concurrently.

12. The method of claim 9 or 10, wherein the agent that antagonizes LIF and the chemotherapeutic agent are administered sequentially.

13. A method of treating a pancreatic cancer in a subject, the method comprising administering to the subject a therapeutic amount of an agent that antagonizes leukemia inhibitory factor (LIF).

14. The method of claim 13, wherein the pancreatic cancer is a pancreatic ductal adenocarcinoma.

15. The method of claim 13 or 14, wherein the agent that antagonizes LIF is an anti-LIF antibody.

16. The method of claim 15, wherein the anti-LIF antibody is a monoclonal antibody.

17. The method of claim 15, wherein the anti-LIF antibody is an antibody fragment selected from the group consisting of a Fab, a F(ab′)₂, and a Fv.

18. The method of any of claims 13-17, wherein the agent that antagonizes LIF is administered orally, intravenously, or intraperitoneally.

19. The method of any of claims 13-18, wherein the agent that antagonizes LIF is administered in combination with a chemotherapeutic agent.

20. The method of claim 19, wherein the chemotherapeutic agent is gemcitabine.

21. The method of claim 19 or 20, wherein the agent that antagonizes LIF and the chemotherapeutic agent are administered concurrently.

22. The method of claim 19 or 20, wherein the agent that antagonizes LIF and the chemotherapeutic agent are administered sequentially.

23. A kit for treating a K-Ras-expressing cancer, the kit comprising:
an agent that antagonizes leukemia inhibitory factor (LIF); and
a chemotherapeutic agent.

24. The kit of claim 23, wherein the agent that antagonizes LIF is an anti-LIF antibody.

25. The kit of claim 24, wherein the anti-LIF antibody is a monoclonal antibody.

26. The kit of any of claims 23-25, wherein the chemotherapeutic agent is gemcitabine.
